(19) United States
(12) Patent Application Publication
(10) Pub. No.: US 2019/0240297 A1
(43) Pub. Date: Aug. 8, 2019

(54) TREATMENT OF INFLAMMATORY BOWEL DISEASE

(71) Applicant: UNIVERSITÉ LAVAL, Québec (CA)

(72) Inventors: Jean SÉVIGNY, Québec (CA); Mabrouka SALEM, Québec (CA)

(21) Appl. No.: 16/336,793

(22) PCT Filed: Sep. 29, 2017
(86) PCT No.: PCT/CA2017/051150

§ 371 Date: Mar. 26, 2019

Related U.S. Application Data
(60) Provisional application No. 62/402,045, filed on Sep. 30, 2016.

(51) Int. Cl. A61K 38/46; A61P 1/00; A61K 31/26; A61P 35/00; G01N 33/50; A61P 29/00; A61K 9/00

(52) U.S. Cl. CPC A61K 38/46; A61P 1/00; A61K 31/26; A61P 35/00; G01N 33/5088; A61P 29/00; G01N 2800/52; A61K 9/0053; C12Y 306/01005; G01N 2333/726; G01N 2800/065; G01N 2800/50; A61K 9/0031

(57) ABSTRACT

The present invention discloses a novel therapeutic axis NTPDase8 enzyme, P2Y₆ and/or P2Y₂ receptors, and provides methods, enzymes and/or antagonists for remedying inflammatory bowel diseases.

### INCORPORATION BY REFERENCE OF SEQUENCE LISTING

The Sequence Listing, which is a part of the present disclosure, includes a computer readable form and a written sequence listing comprising nucleotide and/or amino acid sequences. The sequence listing information recorded in computer readable form is identical to the written sequence listing. The subject matter of the Sequence Listing is incorporated herein by reference in its entirety

### FIELD OF THE INVENTION

The present invention relates to NTPDase8 as a new molecular target for treating inflammatory bowel diseases. Also shown is that P2Y₆ deficiency exerts a protective role on intestinal inflammation development. P2Y₂ deficiency also exerts a protective effect but to a lower extent, whereas NTPDase8 activity regulates the activation of these 2 Intestinal epithelial receptors. Also proposed is a novel therapeutic axis NTPDase8-P2Y₆-P2Y₂ receptors for screening for active molecules, for preventing or treating inflammatory bowel diseases, and thus eventually preventing its end result, GI cancer.

### BACKGROUND OF THE INVENTION

Inflammatory bowel diseases (IBDs) Including a wide spectrum of disorders is characterized by acute or chronic inflammation within the gastrointestinal tract and relapsing of immune system activation followed by marked alterations of several digestive functions (Xu X R, 2014) which leads to abnormal intestinal secretion and marked pain (Lakhan SE, 2010). Intestinal epithelial dysfunction appears to be central in inducing intestinal inflammation, whereas long term chronic inflammation of the GI tract may often result in GI tract cancer.

In fact, intestinal epithelial cells (IEC) respond to pathogens present in extracellular environment by recognizing specific danger signals such as extracellular nucleotides (Kono H, 2008). A few groups suggested that ATP constitute an endogenous danger signal that promotes inflammation (Lister M F, 2007; Riteau N, 2010) by amplifying and sustaining the responses to cytokines. Some lines of evidence support a pivotal role of nucleotides and their signalling in the modulation of immune/inflammatory intestinal cell activities (Antonioli L, 2008, 2013. Grbic, 2008, 2012). In fact, nucleotides such as ATP, UTP and the products of their hydrolysis ADP and UDP respectively activate purinergic receptors P2 which are subdivided into metabotropic P2Y receptors (P2YRs) and ionotropic P2X receptors (P2XRs) on the basis of their signalling properties (Burnstock G, 2007). The activation of these receptors is modulated by members of a family of ectonucleotidases named nucleosides triphosphate diphosphohydrolases (NTPDase), which are important enzymes metabolising nucleotides and terminating purinergic signalling.

Eight members of this family have been identified with different biochemical and structural characteristics (Bigonnesse F, 2004). The first member of this family, NTPDase1, is expressed on lamina propria Infiltrated cells including T cells (particularly T-regulatory cells) (Deaglio S et al. 2007) and it was shown that NTPDase1 mutation or deletion increases susceptibility to inflammatory bowel disease (Friedman D J, 2009). Knowing that the intestinal epithelium plays an important role in regulation and protection of intestine, we hypothesized that such enzymatic activity in the apical surface of the Intestine could regulate induction of intestinal inflammation by reducing the concentration of the nucleotides in the intestinal lumen. But the mechanisms would therefore be different as it would involve the activation of epithelial receptors that would involve different functions. We reported in our previous work that the last member of NTPDases family named, NTPDase8, is detected in jejunum and kidney in RNA level (Bigonnesse F, 2004) but the cellular expression of NTPDase8 was not studied.

This study allows the localisation of the NTPDase8 in the intestine, and the characterisation of its role in intestinal homeostasis and inflammation. The present results show that the NTPDase8 is expressed on intestinal epithelial cells and although it is responsible of a very low level of nucleotides hydrolysis in the entire intestine, its precise localisation makes it a most important regulator of intestinal Inflammation.

### SUMMARY OF THE INVENTION

In a first aspect of the invention, there is provided an NTPDase enzyme for use in the treatment or prevention of chronic inflammatory bowel diseases in a mammal.

In a further aspect, the invention described a method for identifying a candidate compound for treating, reducing or preventing chronic inflammatory bowel diseases in a mammal, the method comprising: contacting a NTPDase1, -3 or -8 enzyme or a cell expressing an NTPDase gene with one or more test compounds; measuring NTPDase1, -3 or -8 gene expression or NTPDase8 enzyme activity in the cell, selecting one of the test compounds that increases the expression or activity relative to NTPDase1, -3 or -8 expression or activity in the cell when compared to a cell or enzyme not contacted with the compound, and testing the one compound as a candidate drug for treating, reducing or preventing chronic inflammatory bowel diseases in the mammal.

According to a further aspect, there is provided a composition comprising an NTPDase enzyme and/or an inhibitor or antagonist of P2Y₆ receptor for use in the treatment or prevention of chronic inflammatory bowel diseases in a mammal.

According to a further aspect, the invention describes a composition comprising apyrase and/or an Inhibitor or antagonist of P2Y₆ receptor for use in the treatment or prevention of chronic inflammatory bowel disease in a mammal.

In accordance with a further aspect, there is provided use of a NTPDase enzyme for the manufacture of a medicament for the treatment or prevention of a chronic inflammatory bowel disease in a mammal.

In accordance with a further aspect, there is provided a method for the treatment or prevention of chronic inflammatory bowel diseases in a mammal, the method comprising administering to said subject an effective amount of a composition comprising NTPDase enzyme.

According to a further aspect, the invention describes a method for the treatment or prevention of chronic inflammatory bowel diseases in a mammal, the method comprising administering to the subject an effective amount of apyrase, in combination with an inhibitor or antagonist of P2Y₆ receptor.

According to a further aspect, the invention describes a method for identifying a risk of a subject to develop a chronic inflammatory bowel disease (IBD), the method comprising: obtaining an intestinal epithelium sample from the subject and measuring expression or enzymatic activity of NTPDase enzyme on a surface thereon; whereby a decreased expression or activity in NTPDase compared to a control level means an increased risk of developing IBD.

In accordance with a further aspect, there is provided a method for monitoring progression of a chronic inflammatory bowel disease in a mammal, the method comprising: measuring NTPDase8 gene or protein expression and/or NTPDase8 protein activity on a lumen surface of a colon cell from the mammal; or measuring a level of expression of P2Y₆ or P2Y₂ receptor on a lumen surface of a colon cell from the mammal; wherein a decrease in NTPDase8 protein and/or activity, or an increase in level or activity of P2Y₆ or P2Y₂ receptor, is Indicative of a progression of said disease, or an increase in NTPDase8, and/or a decrease in level of P2Y₆ or P2Y₂ receptor, is indicative of a remission of the disease, whereby the increase or decrease is compared to a control level from a control population, or to a control time-point data from the mammal.

In accordance with a further aspect, there is provided an Inhibitor or antagonist of P2Y₂ or P2Y₆ receptor, or a combination thereof, for use in the treatment or prevention of chronic inflammatory bowel diseases in a mammal.

In accordance with a further aspect, there is provided a use of an inhibitor or antagonist of P2Y₂ or P2Y₆ receptor, or a combination thereof, for treatment or prevention of chronic inflammatory bowel diseases in a mammal.

In accordance with a further aspect, there is provided a method for treating or preventing chronic inflammatory bowel diseases in a mammal, comprising administering an activation-decreasing dose of an inhibitor or antagonist of P2Y₂ or P2Y₆ receptor, or a combination thereof.

### DETAILED DESCRIPTION OF THE INVENTION

### Description of the Figures

FIGS. 1A-B. NTPDase8 is localised at the apical surface of the mouse and human colon. Immuno-histochemical assay performed on mouse colon section (A) or human colon biopsy (B) stained with mouse (A) or human (B) anti-NTPDase8 antibody. Pre-immune control or an irrelevant IgG2a showed no immunoreactivity.

FIGS. 2A-E. Generation and characterization of Entpd8^{-/-} mice. A. The NTPDase8 gene was replaced by homologous recombination using the cassette ZEN-UB1 which encodes neomycin. B. Genomic DNA was prepared from WT and Entpd8^{-/-} mice and analyzed by PCR. The primers detected a band of 766 pb corresponding to NTPDase8 gene for the WT mouse. C. Detection of NTPDase8 mRNA in WT and Entpd8′ mouse colons by RT-PCR. NTPDase8 RNA is present in the WT colon (PCR product of 240 bp) but absent in Entpd8^{-/-} mice. GAPDH was used as a control for cDNA quality. D. Enzyme histochemistry assay performed on serial tissue sections of colons show ATPase and ADPase activities. ATPase and ADPase activities (100 μM of ATP or ADP respectively) are detected on apical side of WT colonic epithelium by histochemistry whereas no activity Is detected in Entpd8^{-/-} colonic epithelia. Nuclei were counterstained with hematoxylin. Arrows indicate the apical surface of IECs (with or without positive staining). Scale bar: 20 μm. E. Decreased luminal ATPase activity in colon of Entpd8^{-/-} mice. ATP activity in the luminal fluid was measured by malachite green method 15 min post injection of PBS with or without ATP (1.5 mM) Into the lumen of the colon loop. Data are the mean±S.E.M. of 3 independent experiments, each with groups of 4 mice.

FIGS. 3A-D. Exacerbated acute dextran sodium sulfate (DSS)-induced colitis in NTPDase8 deficient mice. A. Disease activity Index (DAI) of WT, Entpd8^{-/-} and Entpd1^{-/-} (used as a control) mice. n=20/group. B. colon length from DSS-treated WT and Entpd8^{-/-} mice at day 7 of acute DSS-induced colitis. C. Representative histological photos of hematoxylin and eosin (H&E)-stained colon sections from the indicated groups at day 7 of acute DSS colitis. Scale bar: 20 μm. D. Histological scoring of sections from WT and Entpd8^{-/-} DSS treated colons. The data shown are the mean±S.E.M, n=12. * p<0.05; ** p<0.01, *** p<0.001 compared to non-treated mice. § p<0.05; §§ p<0.01 compared to DSS-treated WT.

FIGS. 4A-B. Increased apoptosis in colonic crypts of DSS-treated Entpd8^{-/-} mice. A. Apoptosis was evaluated on paraffin colon sections using anti-caspase-3 antibody. Representative images of colonic sections of 18 mice are shown. Apoptotic cells can be seen in a brown color. Nuclei were counterstained with hematoxylin. Scale bar: 20 μm. B. Quantification of positive cells was done by Image J software. *** p<0.001.

FIGS. 5A-C. DSS treatment increases the infiltration of immune cells in colon of Entpd8^{-/-} more than in WT mice. Quantification by Image J software of macrophage (A) and lymphocyte (B) infiltration evaluated by immunohistochemistry with F4/80 or anti-CD3 antibody, respectively. C. Measurement of myeloperoxidase activity (MPO) in the homogenate of the entire colons as an indicator of polymorphonuclear leucocytes (neutrophils). Values are expressed as the mean±S.E.M. of 18 mice. *** p<0.001.

FIG. 6. The presence of NTPDase8 affects the profile of cytokine and chemokines. Expression of mRNA level for cytokine and chemokines in colon of Entpd8^{-/-} and WT mice was analyzed by qRT-PCR. Data were normalized with GAPDH mRNA levels. The data represent are the mean±S.E.M. of 16 mice. ** p<0.01; *** p<0.001.

FIGS. 7A-E. Characterization of primary IEC cultures. Intestinal crypt containing proliferative stem cells were isolated, treated with Collagenase and cultured for 4 days in presence of growth factors to form a monolayer. A-C. IEC were analyzed by qRT-PCR for the expression of the epithelial cell marker villin (A), P2Y receptors (B) and ectonucleotidases (C). Data are expressed as quantity of indicated mRNA expression normalized to GAPDH. D, E. Enzyme activity assays of Intact IEC (D) and protein extract from lysed IEC (E) for ATPase and ADPase activities. Data presented are the mean±S.E.M of three independent groups of 3 mice each group. ** p<0.01.

FIGS. 8A-F. Mice deficient in P2Y₆ receptor in IEC are protected from DSS-induced colitis. A. Analysis of chimerism as the percentage of GFP+ cells (hematopoletic cells from the donor) compared to total hematopoietic cells number (CD45+ cells) by FACS on peripheral blood from the chimeric mice generated. Data are shown as the mean±S.E.M. B, C. WT and P2ry6^{-/-} mice were irradiated then transplanted with bone marrow cells. After 8 weeks, DSS (3%) was added to the drinking water for 7 days. The DAI (B) and colon length (C) at day seven averaged for the animals of each group are presented (10 mice/group). D. MPO activity was assessed in the homogenate of the entire colons. E. Representative H&E staining of colons from chimeras. Bar scale: 20 μm. F. Expression of mRNA for cytokines and chemokines in colons was analyzed by qRT-PCR. Data were normalized to GAPDH mRNA levels. Data presented are the mean±S.E.M. of 16 mice. * p<0.05; ** p<0.01; *** p<0.001 compared to WT in P2ry6^{-/-} mice. φp<0.05, φφp<0.01, φφφp<0.001 compared with their respective mice that received water instead of DSS.

FIG. 9. P2ry2^{-/-} mice are less susceptible to DSS-induced colitis. The mean !S.E.M. of the DAI measured for WT and P2ry2^{-/-} mice treated for 7 days with DSS is shown, n=9. * p<0.05; ** p<0.01; *** p<0.001, P2ry2^{-/-} DSS-treated mice compared with DSS-treated WT mice.

FIGS. 10A-D. Inhibition of the P2Y₆ receptor attenuates DSS-induced colitis. During treatment with DSS, Entpd8^{-/-} (A, B) or WT (C, D) mice were administrated daily intrarectally with the P2Y₆ antagonist MRS 2578 (10 μM) or its vehicle (DMSO) from day 2 to day 7. A, C. DAI was recorded daily 8 hours after the intrarectal injection. B, D. At day 7, 4 hours after MRS 2578 or DMSO injection, mice received Dextran-FITC by gavage. Intestinal permeability was assessed another 4 hours later by evaluating the presence of FITC in the plasma collected after sacrifice. The mean±S.E.M. of 5 mice per group are shown. * p<0.05; ** p<0.01; *** p<0.001. Arrow indicates the first MRS 2578 or vehicle injection.

FIGS. 11A-D. Degradation of nucleotides during DSS-induced colitis protects WT mice from intestinal inflammation. During treatment with DSS, WT mice were administrated intrarectally with apyrase (4 U/mice) or its vehicle (PBS) daily from day 2 to day 7 (A, B) or from day 5 to day 7 (C, D). A, C. DAI was recorded daily 8 hours after the intrarectal injection. B, D. At day 7, 4 hours post injection, mice received Dextran-FITC by gavage. Intestinal permeability was assessed 4 hours later by evaluating the presence of FITC in the plasma collected after sacrifice. Representative data of the mean±S.E.M. of 5 mice are shown. * p<0.05; ** p<0.01; *** p<0.001. Arrow indicates the beginning of apyrase or vehicle injection.

FIGS. 12A-C. Characterization of primary human IEC culture. (A) Human crypts were isolated directly from colon tissue, then half was recuperated in trizol to extract the RNA. The other part was used to differentiate epithelial cells to obtain an epithelial monolayer. For this, crypts were incubated with collagenase XI then cultured in presence of growth factors and Y-27 (as an inhibitor of anoikis). After 48 hours, fresh medium was added to cells without Y-27 for 2 days to obtain a differentiated monolayer. Both fraction (crypts and differentiated IEC) were analyzed by qPCR for the markers of differentiation villin and ALPi (A). (B,C) The IEC monolayer was analyzed by qPCR for ectonucleotidases (B) and P2Y receptors (C) mRNA expression. Experiment was done with crypts from the colon of 1 patient (3 wells per condition).

FIGS. 13A-B. UDP-activated P2Y₆ receptor increases KC and CXCL8 expression in murine and human differentiated monolayers, respectively. Crypts from the colon of WT and Entpd8^{-/-} mice or from human colons were differentiated into a confluent epithelial cell monolayer. Obtained IEC were stimulated with UDP (100 μM) for 5 hours then mRNA encoding KC (A; mouse) or CXCL8 (B; human) was measured by qPCR. Values presented are the mean±SEM of 2 groups of 3 mice each (A) or 1 patient (B). *** p<0.001 compared to the untreated respective control (CTL). ### p<0.001 compared to WT cells in the same condition. φφφp<0.001 compared to untreated control cells (CTL).

FIGS. 14A-B. Blocking P2Y₆ receptors during DSS treatment results in decreasing epithelial permeability and chemokines secretion. (A) IEC were grown on permeable supports for 4 days, stimulated with DSS 3% for 24 hours. Six hours later MRS 2578 (10 μM) was added for the last 18 hours. The permeability was evaluated by adding FITC-dextran in the apical chamber of permeable support at the end of the 24 hrs. Four hours later, FITC was measured in the supernatant of basolateral chamber. 3 groups with 3 mice each. Values presented are the mean±SEM. (B). The experiment was performed as described above. After 24 hours of DSS stimulation in absence/presence of MRS 2578, supernatant from the basolateral chamber was collected and KC was measured by ELISA. 1 group of 3 mice. * p<0.05; ** p<0.01; *** p<0.001 compared to respective control. ### p<0.001 compared to WT in the same condition. φp<0.05; φp<0.01 compared to same group with DSS treatment.

### ABBREVIATIONS AND DEFINITIONS

### Definitions

The term “about” as used herein refers to a margin of + or −10% of the number indicated. For sake of precision, the term about when used in conjunction with, for example: 90% means 90%+/−9% i.e. from 81% to 99%. More precisely, the term about refers to + or −5% of the number indicated, where for example: 90% means 90%+/−4.5% i.e. from 86.5% to 94.5%.

As used herein the singular forms “a”, “and”, and “the” include plural referents unless the context dearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, un-recited elements or method steps.

As used in this specification, the terms “control”, “control population” or “control subject” mean an individual or a population of mammals such as humans, characterized by a lack of inflammation symptoms of IBD, or particularly, a population of subjects having undergone a colonoscopy and established healthiness of the colon mucosa.

As used in this specification, the terms “inflammatory bowel disease” or “IBD” means several diseases associated with inflammation of the small intestine, large Intestine (colon), rectum or anus (anal sphincter), and may particularly include ulcerative colitis and Crohn's disease, including proctitis in both cases. As used in the present context, the term “IBD” also includes GI tract cancer, which is a likely result of GI tract inflammation.

As used in this specification, the terms “gastrointestinal tract” or “GI tract” mean the small intestine, large intestine (colon), rectum or anus (anal sphincter).

As used herein, the term “NTPDase enzyme” means an NTPDase1, an NTPDase3, an NTPDase8, or derivatives thereof such as, as non-limiting examples: alkaline and acid phosphatases, NTPDases or other nucleotidases from insects, other vertebrate, bacteria, or plants generally called apyrases as well as mutants of NTPDase1 such as the soluble form generated by the groups of Marcus and Pinsky (Buergler J M, 2005; Gayle RB 3rd, 1998), NTPDase3 derivatives generated by Kiriey's group and also, importantly, the NTPDase mutant from the group of Ridong Chen and colleagues (US2009/0035291 or www.ncbi.nlm.nih.gov/pubmed/25100739), or all other forms listed in the following Table 1:

TABLE 1
Summary of ectoenzymes potentially involved in the modulation of P2 and P1 receptor signalling

| | | | | | | | |
|---|---|---|---|---|---|---|---|
| | | Other | Enzyme | Enzymatic | | | Functions or potential |
| Ectoenzyme | Gene* | alias | Class | Reaction | Substrates | Inhibitors | functions |

Nucleoside triphosphate diphosphohydrolases (NTPDases)

| | | | | | | | |
|---|---|---|---|---|---|---|---|
| NTPDase1 | ENTPD1 | CD39, | EC 3.6.1.5 | NTP → NDP + Pᵢ | NTP, NDP | NaN₃, 8-BuS- | Prevent P2Y₁ and P2X1 |
| | | apyrase, | | NDP → NMP + Pᵢ | | ATP, | desensitization |
| | | ATPDase | | | | ARL 67156, | Terminate P2 signalling |
| | | | | | | POM-1, | (P2Y₂, P2Y₆, P2X7, etc) |
| | | | | | | ticlopidine, | Favor adenosine generation |
| | | | | | | clopidogrel, | |
| NTPDase2 | ENTPD2 | CD39L1, | | | NTP | PSB-6426, | Termination of P2 signalling |
| | | ecto-ATPase | | | | POM-1 | Switch P2 activation |
| NTPDase3 | ENTPD3 | CD39L3, HB6 | | | NTP, NDP | ARL 67156, | Termination of P2 signalling |
| | | | | | | hN3-H10s | Transient switch of |
| | | | | | | | P2 activation |
| NTPDase4 | ENTPD4 | UDPase, | | | | | |
| | | LALP70 | | | | | |
| NTPDase5 | ENTPD5 | CD39L4, | | | NDP | | Termination of P2 signalling |
| | | PCPH | | | | | |
| NTPDase6 | ENTPD6 | CD39L2 | | | UDP, ADP, UTP | | Termination of P2 signalling |
| NTPDase7 | ENTPD7 | LALP1 | | | | | |
| NTPDase8 | ENTP08 | Hepatic | | | NTP, NDP | | Termination of P2 signalling |
| | | ATPDase | | | | | Transient switch of |
| | | | | | | | P2 activation |

Nucleotide pyrophosphatases/phosphodiesterases (NPPs)

| | | | | | | | |
|---|---|---|---|---|---|---|---|
| NPP1 | ENPP1 | CD203a, | EC 3.1.4.1 | NTP → NMP + PPᵢ | ATP, NpₙN, pNP- | ARL 67156 | Termination of P2 signalling |
| | | PC-1 | EC 3.6.1.9 | NDP → NMP + Pᵢ | TMP, NAD⁺ | Me-Ap₅A-Me, | Favor P2 activation (from |
| NPP2 | ENPP2 | Autotaxin | | NpₙN → NMP + | | Me-dAp₅dA- | dinucleotides' hydrolysis) |
| | | | | Np₍ₙ₋₁₎ | | Me | Favor adenosine generation |
| NPP3 | ENPP3 | CD203c, | | NAD⁺ → AMP + | ATP, NpₙN, pNP- | Me-Ap₅A-Me, | |
| | | GP130^{RB13-6}, | | nicotinamide | TMP, LPC, SPC | Me-dAp₅dA- | |
| | | B10 | | ribosyl-P | | Me | |
| | | | | 3′-5′-NMPc → | ATP, NpₙN, | | |
| | | | | NMP | pNP-TMP | | |

Phosphatases (ACP & ALP)

| | | | | | | | |
|---|---|---|---|---|---|---|---|
| PAP | ACPP | ACP3 | EC 3.6.1.2 | NMP → | NMP, pNP-P, | L-(+)-tartrate, | Activate P1 receptors |
| | | | | nucleoside + Pᵢ | various | NaF, | |
| | | | | | phosphorylated | α-BzNBz | |
| | | | | | molecules | Phosphonic | |
| | | | | | (alkaloid, lipid, | acid | |
| | | | | | protein, sugar) | | |
| OcAP/TrAP | ACP5 | | | NTP → NDP + Pᵢ | NTP, NDP, NMP, | | Termination of P2 signalling |
| | | | | NDP → NMP + Pᵢ | pNP-P, various | | Activation of P1 receptors |
| | | | | NMP → nucleoside + | phosphorylated | | |
| | | | | Pᵢ | molecules | | |
| | | | | Molecule-P → | (alkaloid, lipid, | | |
| | | | | molecule + Pᵢ | protein, sugar) | | |
| TNAP | ALPL | akp-2 | EC 3.6.1.1 | NTP → NDP + Pᵢ | NTP, NDP, NMP, | Levamisole | Termination of P2 signalling |
| | | | | NDP → NMP + Pᵢ | pNP-P, cAMP, | | Activate P1 receptors |
| | | | | NMP → nucleoside + | various | | |
| | | | | Pᵢ | phosphorylated | | |
| | | | | Molecule-P → | molecules | | |
| | | | | molecule + Pᵢ | (alkaloid, lipid, | | |
| | | | | | protein, sugar) | | |

Others which are soluble enzymes and not ectoenzymes

| | | | | |
|---|---|---|---|---|
| soluble | CANT1 | | NTP and NDP | References: www.ncbi.nlm. |
| calcium- | | | | nih.gov/pubmed/12234496 |
| activated | | | | Smith T., Hicks-Berger |
| nucleotidase | | | | C., Kim S., Kirley T. Arch. |
| (SCAN) also | | | | Biochem. Biophys. 406: |
| named | | | | 105-115(2002) |
| Ca(2+)- | | | | Yang M., Kirley T.L. |
| dependent | | | | Biochemistry |
| endoplasmic | | | | 43: 9185-9194(2004) |
| reticulum | | | | https://en.wikipedia.org/ |
| nucleoside | | | | wiki/CANT1 Failer BU1, |
| diphosphatase. | | | | Braun N. Zimmermann |
| | | | | H.J Biol Chem. |
| | | | | 2002 Oct 4; 277(40): |
| | | | | 36978-86 |
| | | | | Cloning, expression, and |
| | | | | functional characterization |
| | | | | of a Ca(2+)-dependent |
| | | | | endoplasmic reticulum |
| | | | | nucleoside diphosphatase. |
| Apyrase from | | As for NTPDases | NTP and NDP | |
| potato and | | | | |
| other plants | | | | |

(from Kukulski et al. 2011 and Beaudoin et al. 1996)

### DETAILED DESCRIPTION OF PARTICULAR EMBODIMENTS

Herein presented is a demonstration that NTPDase8 is localized on the apical side of colonic epithelium and its deletion exacerbates intestinal inflammation by promoting the activation of P2Y₆ receptor expressed on intestinal epithelial cells (IEC).

Also presented herein presented is a demonstration that NTPDase8 deletion exacerbates intestinal Inflammation by promoting the activation of P2Y₂ receptor expressed on intestinal epithelial cells (IEC).

Hence, there is herein proposed a novel therapeutic axis NTPDase8-P2Y₆-P2Y₂ receptors for remedying inflammatory bowel diseases.

### Screening Method for Active Compounds

According to a particular embodiment, there is provided a method for identifying a candidate compound for treating, reducing or preventing chronic inflammatory bowel diseases in a mammal, the method comprising: contacting a NTPDase8 enzyme or a cell expressing a NTPDase8 gene with one or more compounds; measuring NTPDase8 gene expression or NTPDase8 protein activity in the cell, identifying one of said compounds that increases the expression or activity relative to NTPDase8 expression or activity in a cell when compared to a cell or enzyme not contacted with the compound, and using the compound as a candidate drug for treating, reducing or preventing chronic inflammatory bowel diseases in the mammal.

### Screening Method for Risk Assessment of Developing Disease

According to a particular embodiment, there is provided a method for identifying a risk of a subject to develop a chronic Inflammatory bowel disease, the method comprising: obtaining an intestinal epithelium sample from the subject; and measuring expression of NTPDase enzyme, P2Y₆ receptor or P2Y₂ receptor on a surface thereon; whereby a low expression in NTPDase8 enzyme and/or a low level of its activity, high P2Y₆ receptor number or activation or high P2Y₂ receptor number or activation means/is indicative of/or suggests an increased risk of developing IBD.

According to a particular embodiment, there is provided a method for monitoring progression of a chronic inflammatory bowel disease in a mammal, the method comprising: measuring NTPDase8 gene expression or NTPDase8 protein activity in a colon cell from the mammal, or measuring a level of expression of P2Y₆ or P2Y₂ receptor on a surface of a colon cell from the mammal, wherein a decrease in NTPDase8 or increase in level of P2Y₆ or P2Y₂ receptor is indicative of/means/or suggests a progression of the disease, or an increase in NTPDase8 and/or decrease in level of P2Y₆ or P2Y₂ receptor is indicative of/means/or suggests a remission of the disease, whereby the Increase or decrease is compared to a control level from a control population, or from a control point of the mammal.

### Uses of NTPDase Enzymes

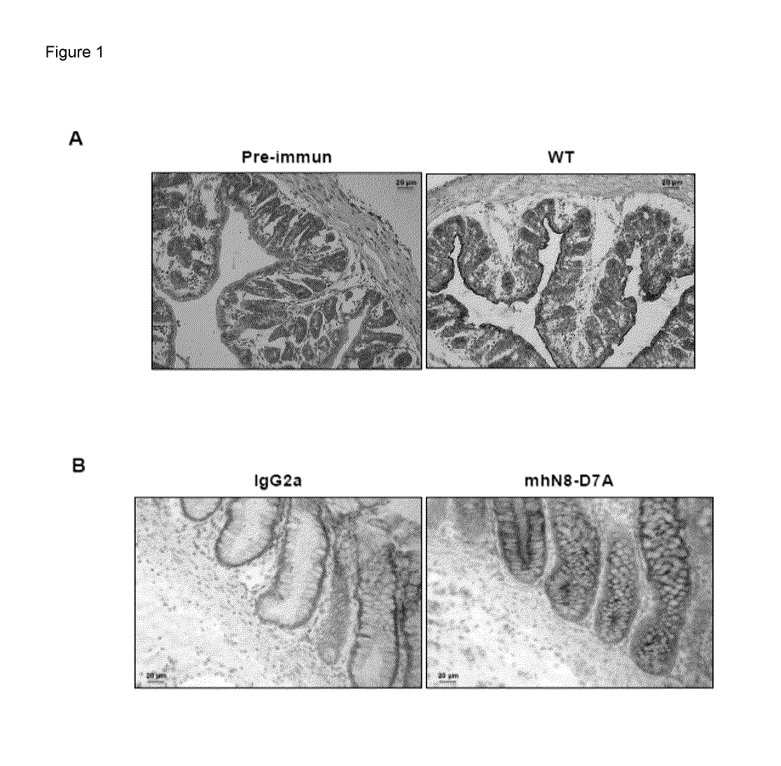

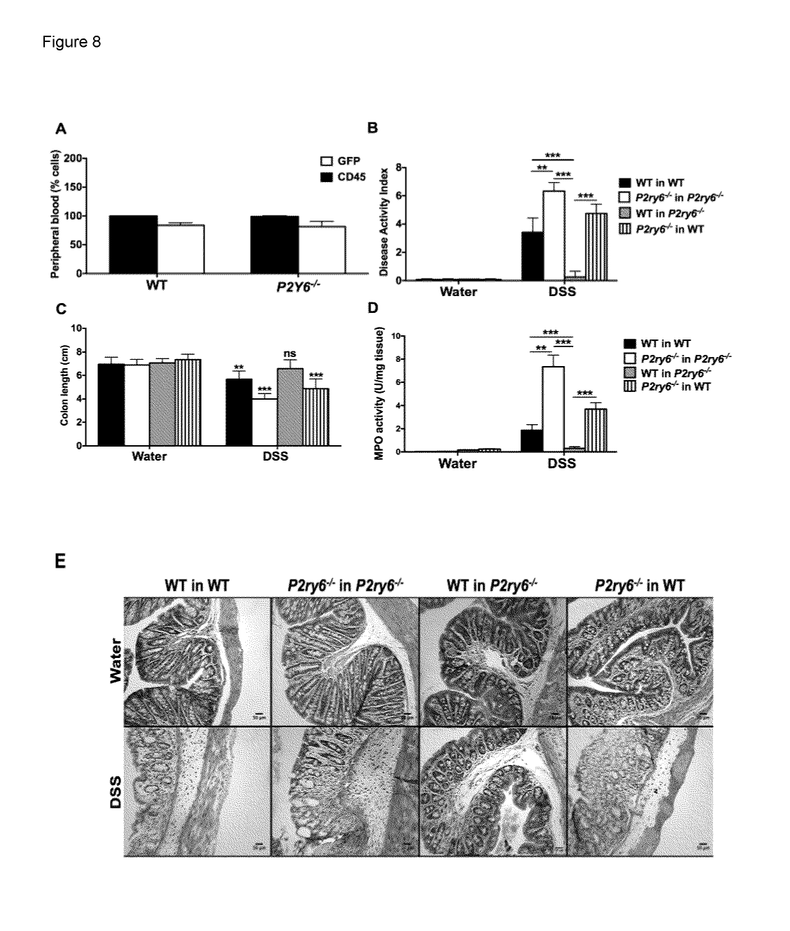

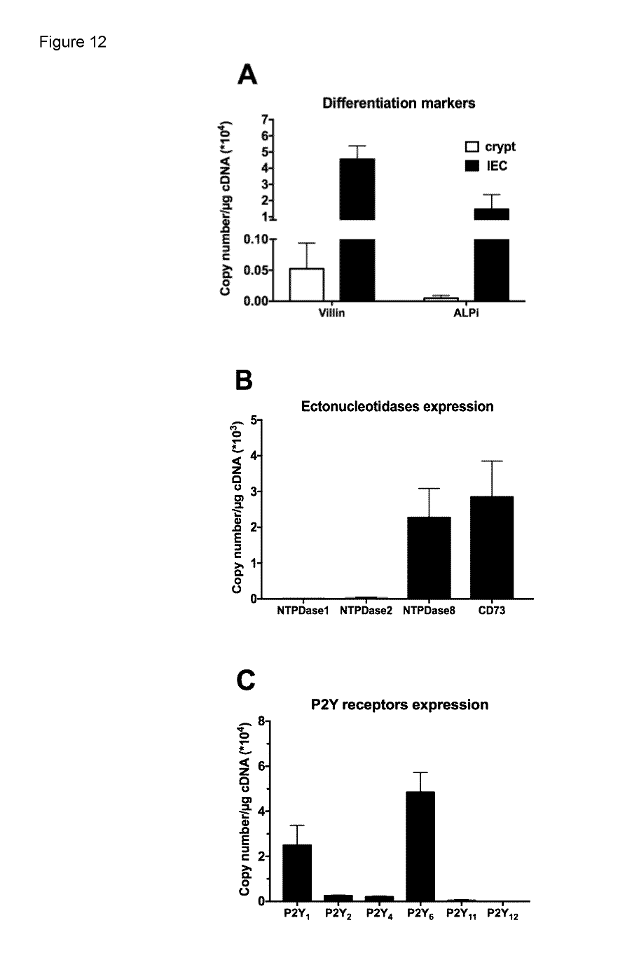

Herein, we characterized the members of purinergic signaling in primary IEC isolated from colon samples and showed that murine IEC expressed predominantly the ectonucleotidase NTPDase8 (FIGS. 1A and 7C). We later detected the presence of NTPDase8 on human IEC (FIGS. 1B and 12B).

Therefore, in accordance with a particular embodiment, there is provided a use of a NTPDase enzyme for the manufacture of a medicament for the treatment or prevention of chronic inflammatory bowel disease in a mammal.

In accordance with a particular embodiment, there is provided a NTPDase enzyme for use in the treatment or prevention of chronic inflammatory bowel disease in a mammal.

In accordance with a particular embodiment of the invention, the NTPDase enzyme is chosen from: an NTPDase1, an NTPDase3, an NTPDase8, or a derivative thereof, such as the ones defined in the definition section, paragraph [0038] and/or in Table 1. Particularly, the NTPDase enzyme is chosen from: NTPDase1, NTPDase3, or NTPDase8. More particularly, the enzyme is NTPDase8.

In accordance with a particular embodiment, the NTPDase enzyme of the invention is present (or administered) in a concentration sufficient to decrease a concentration of a nucleoside triphosphate or diphosphate in a colon lumen of the mammal.

Particularly, the nucleoside triphosphate or diphosphate is selected from: UTP, UDP, ADP and ATP. More particularly, the nucleoside triphosphate or diphosphate is selected from: UTP. UDP and ATP.

### Receptor P2Y₆

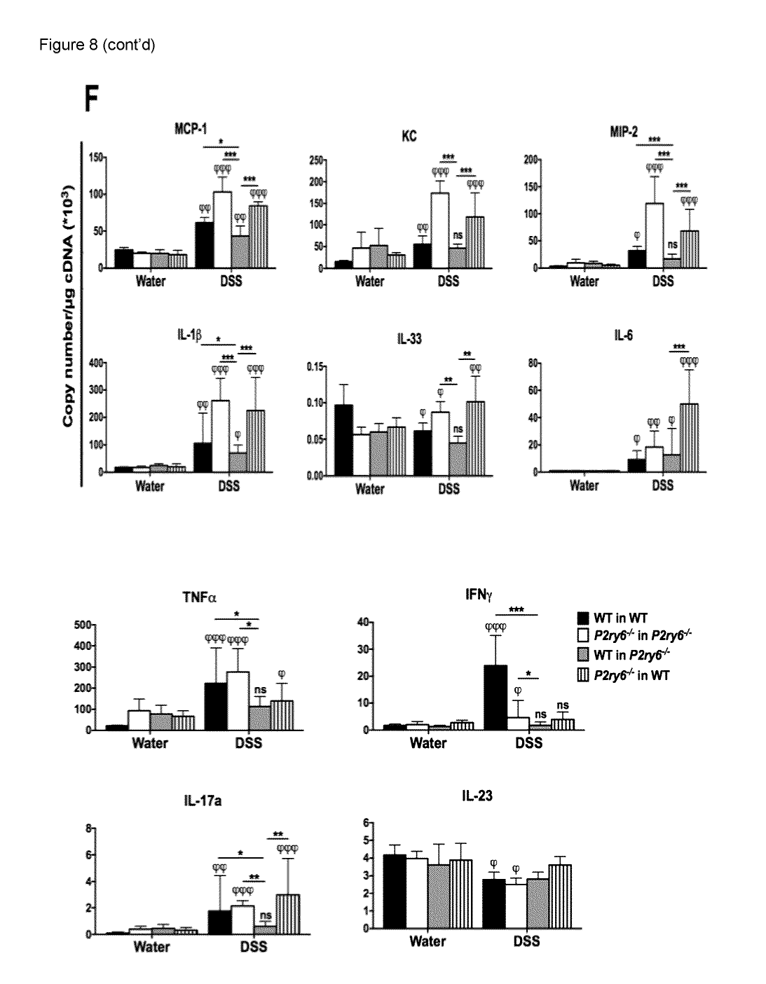

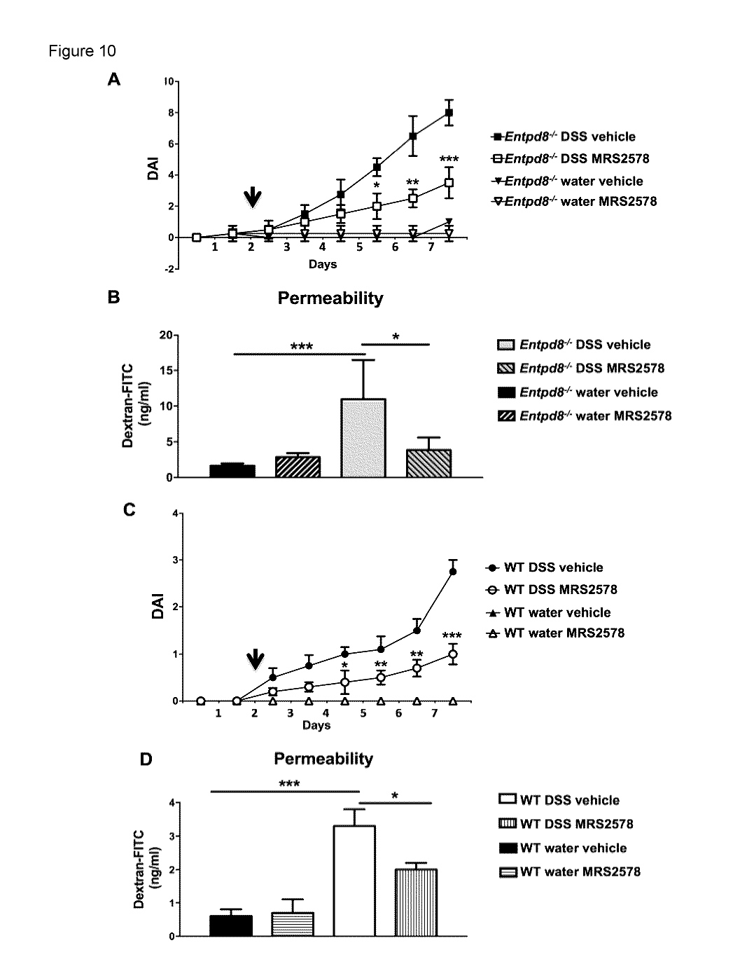

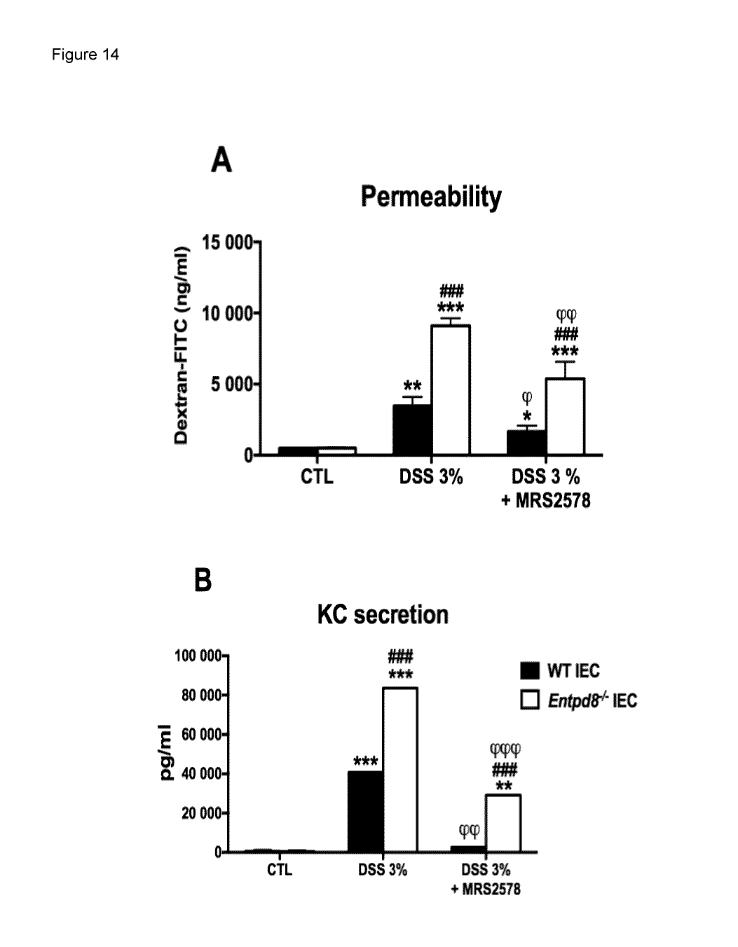

Also showed is that P2Y₆ deficiency in murine IEC exerts a protective role on intestinal inflammation development (FIG. 8). In addition, the present results also show that a P2Y₆ antagonist exerts a similar effect (FIG. 10). Note that we have also detected the presence of NTPDase8 and P2Y₆ on human IEC (FIGS. 1B, 12B and 12C). We later verified that blocking P2Y₆ could have protective effects in human cells (FIG. 14).

Therefore, according to a particular embodiment, there is provided a use of NTPDase8 medicament as defined herein, optionally in combination with an inhibitor of a P2Y₆ receptor such as a molecule that prevents the binding of its natural ligand or that reduces P2Y₆ expression.

Alternatively, according to a particular embodiment, there is provided a use of an Inhibitor of a P2Y₆ receptor in the treatment or prevention of chronic Inflammatory bowel disease in a mammal.

In accordance with a particular embodiment of the Invention, the P2Y₆ receptor inhibitor is chosen from: MRS 2567, MRS 2575, MRS 2578 (a specific and selective antagonist: www.ncbi.nlm.nih.gov/pubmed/28527783), TIM-38 (a specific and selective antagonist), Reactive Blue 2 (RB2), suramin and suramin derivatives.

### Receptor P2Y₂

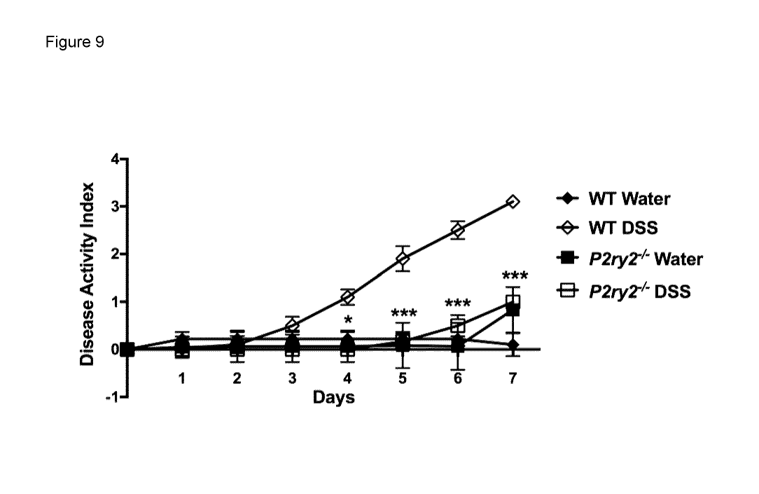

Also presented herein is the fact that full deletion of P2Y₂ receptor also protects mice, to some extent, from DSS-induced colitis (FIG. 9).

In accordance with a particular embodiment, there is therefore provided a use of NTPDase8 medicament as defined herein, in combination with an inhibitor of a P2Y₂ receptor such as a molecule that prevents the binding of its natural ligand or by reducing P2Y₂ expression.

Alternatively, according to a particular embodiment, there is provided a use of an Inhibitor of a P2Y₂ receptor in the treatment or prevention of chronic inflammatory bowel disease in a mammal.

In accordance with a particular embodiment of the invention, the P2Y₂ receptor inhibitor is chosen from: the thiouracil derivative AR C118925 (a specific and selective antagonist), Reactive Blue 2 (RB2), suramin and suramin derivatives.

### Method of Treatment of IBD

In accordance with a particular embodiment, there is provided a method for the treatment or prevention of chronic inflammatory bowel disease in a mammal, the method comprising administering to the subject a composition comprising NTPDase enzyme. Particularly, the NTPDase enzyme is in a concentration sufficient to decrease the levels of the nucleotide: ATP, ADP, UTP, or UDP In the lumen of the intestine (colon). These nucleotides are the natural ligands of P2Y₂ and P2Y₆ receptors.

In accordance with a particular embodiment of the invention, the NTPDase enzyme is chosen from: an NTPDase1, an NTPDase3, an NTPDase8, or a derivative thereof, such as the ones defined in the definition section [0038], and/or in Table 1. Particularly, the NTPDase enzyme is NTPDase1, NTPDase3, or NTPDase8. More particularly the NTPDase enzyme is NTPDase8.

Particularly, the NTPDase or the composition is administered alone, or in combination with an inhibitor of P2Y₆ receptor, and/or in combination with an inhibitor of P2Y₂ receptor.

In accordance with an alternative embodiment of the invention, there is provided a method for the treatment or prevention of chronic inflammatory bowel disease in a mammal, the method comprising administering to a subject an Inhibitor of P2Y₂ receptor or an inhibitor or P2Y₆ receptor. Particularly, the inhibitor of P2Y₆ receptor is administered alone, and/or in combination with an inhibitor of P2Y₂ receptor and/or an NTPDase enzyme.

Alternatively, the inhibitor of P2Y₂ receptor is administered alone, and/or in combination with an inhibitor of P2Y₆ receptor and/or an NTPDase enzyme.

### Therapeutic Indications

According to a further aspect, the disease or condition targeted by the present invention refer particularly to inflammatory bowel disease, selected from, for example: Crohn's disease, ulcerative colitis, and its end result, GI tract cancer.

### Mode of Administration

According to a further aspect, the NTPDase enzyme, receptor inhibitor, compositions, formulation, medicament, or combination, is formulated for oral, enteral, or intrarectal administration.

According to a particular embodiment, the method of treatment comprises intrarectal administration of the NTPDase enzyme, formulation, medicament, or its combination with P2Y₂ and/or P2Y₆ receptor antagonist and/or apyrase.

The following examples are put forth so as to provide those of ordinary skill in the art with a complete disclosure and description of how to make and use the present invention, and are not intended to limit the scope of what the inventors regard as their Invention nor are they intended to represent that the experiments below are all or the only experiments performed. Efforts have been made to ensure accuracy with respect to numbers used (e.g. amounts, temperature. etc.) but some experimental errors and deviations should be accounted for. Unless indicated otherwise, parts are parts by weight, molecular weight is weight average molecular weight, temperature is in degrees Centigrade, and pressure is at or near atmospheric.

### EXAMPLES

### Example 1

### Materials and Methods

### Material

Dextran Sulfate Sodium (DSS) and K₂HPO₄ were purchased from Acros organics. KH₂PO₄ was obtained from EMD Millipore. O-dianisidine hydrochloride, H₂O₂, Noggin, MRS 2578 and apyrase grade VII were purchased from sigma (St. Louis, Mo., USA). Fetal Bovine Serum (FBS) was from Wisent (St-Bruno, Canada), Trizol from Invitrogen (Carlsbad, Calif., USA). mrEGF, R-Spondin and anti-caspase3 antibody were obtained from R&D Systems (Minneapolis, USA), Tgo DNA polymerase, Syber Green and DNasel from Roche (ON, Canada). Anti-CD3 antibody, biotinylated goat anti-guinea pig and goat anti-rabbit secondary antibodies were from Abcam. Mouse anti-NTPDase8 mN8-2_{c}-15 and human anti-NTPDase8 (mhN8-D7As) were generated in our laboratory and can be obtained from www.ectonucleotidases-ab.com. Direct PCR Lysis Reagen from VIAGEN biotech.

### Generation of Entpd8 Deficient Mice

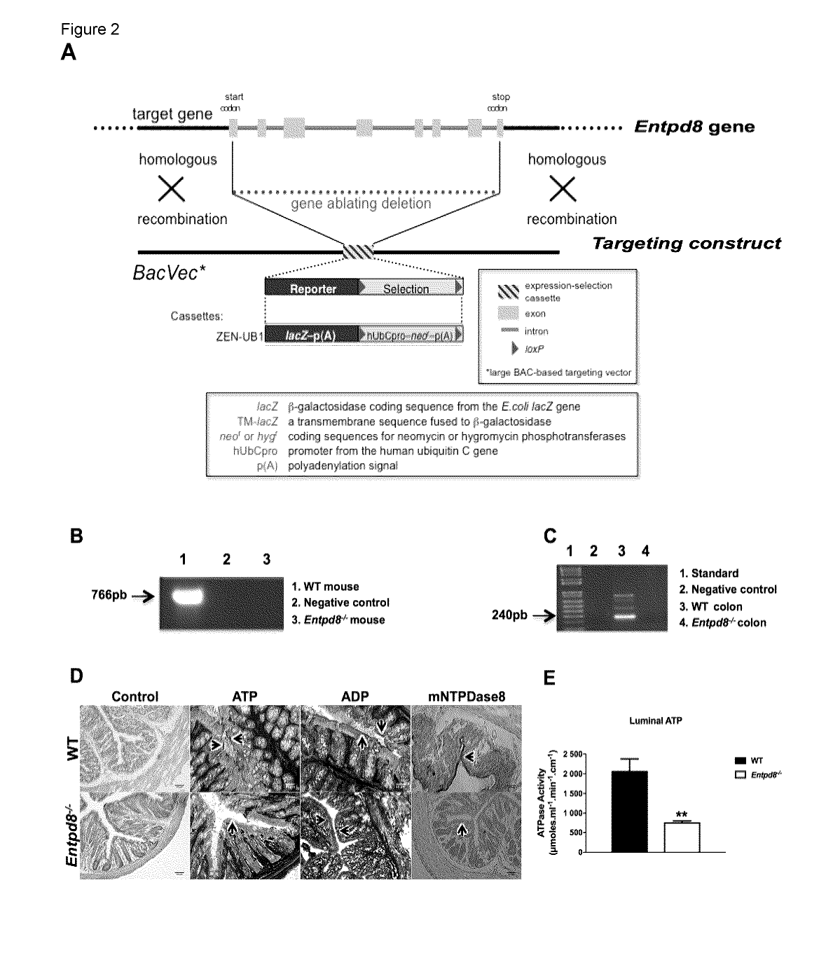

Embryonic stem (ES) cells deficient for Entpd8 gene were constructed using the cassette ZENUB1 by homologous recombination for the Knock-Out Mouse Project (KOMP Repository, University of California) (FIG. 2A). The transformed ES cells were injected by electroporation in blastocysts derived from B6(Cg)-Tyr c-2j/J mice in Jackson Laboratory. Homologous recombination was confirmed by PCR using primers chosen from the sequence of NTPDase8 and neomycin-resistant genes:

NTPDase8gene:
Forward primer:
(SEQ ID NO. 1)
TGT AAG GGC CAG AAG GAT TG;
NTPDase8 gene:
Reverse primer:
(SEQ ID NO. 2)
GTA CAG ACC CGA GGC ACA GT;
neomycin-resistant gene:
Forward primer:
(SEQ ID NO. 3)
TCATTCTCAGTATTGTTTTGCC;
neomycin-resistant gene:
Reverse primer:
(SEQ ID NO. 4)
CAGGCATCTAGGATCTGCTC.

The chimeras generated were bred with C57BL/6 mice for 4 generation then heterozygous Entpd8 gene-disrupted mice were interbred to produce mice with homozygous deletions.

### Animals

All experiments were conducted in accordance with the guidelines of the Canadian Council on Animal Care, and protocols were approved by the Animal Care Committees of Université Laval. Adult male C57Bl/6 mice (Charles River, Canada) Wild Type (WT), Entpd8 Knock out (Entpd8^{-/-}), P2ry2^{-/-} and P2ry6^{-/-} (8-12 weeks old) were bred in our laboratory and were used for all experiments.

### Induction and Clinical Assessment of Experimental (Acute) Colitis

All mice were treated or not (control mice) with 3% (w/v) DSS (molecular weight: 42 kDa) added to the drinking water for 7 days. Disease activity index (DAI) was evaluated daily by scoring percent of weight loss, intestinal bleeding (no blood, presence of blood), and stool consistency (normal stool, loose stool, or diarrhea) as previously described (Berberat P, 2005).

### Histological Assessment of Colitis

Colon specimens were fixed in paraformaldehyde (PFA) 4% for 24 h at 4° C. then embedded in paraffin. Five-micrometer tissue sections were stained with hematoxylin & eosin (H&E) then examined for histological analysis as previously described using as criteria the inflammatory cell infiltration, crypt damage and severity of ulceration using a scale of 0-3 (Laroui H, 2012).

### Colonic Myeloperoxidase Activity

The activity of the enzyme myeloperoxidase (MPO) was determined in colonic mucosa according to a previously described technique (Friedman D J, 2009). Briefly, colon samples were weighed, suspended in 50 mM potassium phosphate buffer, pH 6.0, containing 0.5% hexadecyltrimethylammonlum bromide (HTAB), homogenized by sonication then centrifuged 20 min at 20,000×g. The reaction was started by incubating the supernatant in 1 mg/mL o-dianisidine dihydrochloride, and 0.0005% (vol/vol) hydrogen peroxide. The change in absorbance at 450 nm was measured every 30 seconds over 3 minutes. MPO was expressed in units per milligram of tissue, where 1 unit corresponds to the activity required to degrade 1 mol H₂O₂/min/mL at 24′C.

### Enzyme Histochemistry and Immunohistochemistry

For enzyme histochemistry, mice were perfused with PFA 4%, tissue samples were excised, immersed in sucrose then included in O.C.T. freezing medium (Tissue-Tek®, Sakura Finetk, Torrance, Calif.) and snap-frozen in isopentane in dry ice and stored at -80° C. until use. Sections of 6 μm were prepared and routinely fixed in 4% PFA. Ectonucleotidase activities in colon sections were localized using the Wachstein/Meisel lead phosphate precipitation method as described before (Martin-Satué M, 2009). Briefly, fixed tissue sections were pre-incubated for 30 min at 25° C. in Tris-maleate buffer (2 mM CaCl₂, 250 mM sucrose, 50 mM Tris-maleate, pH 7.4) supplemented with 2.5 mM levamisole as an alkaline phosphatase inhibitor. The reaction was initiated by the addition of the substrate (100 μM of ATP or ADP) in the same buffer supplemented with 5 mM MnCl₂, 2 mM Pb(NO₃)₂ and 3% Dextran T-250 (w/v) and incubated for 3 hours at 37° C. For control experiments, substrate was omitted. Reaction products were revealed as a rust color by incubating the tissue sections with 1% (NH₄)₂S (v/v) for 1 min. Sections were counterstained with aqueous hematoxylin, mounted in mowiol medium and photographed under a BX51 Olympus microscope.

For immunohistochemistry, collected mice tissues were fixed in 4% PFA and embedded in paraffin or fixed in acetone/formalin and embedded in Tissue-Tek O.C.T. compound. Tissue sections were incubated at 4° C. for 18 h with the indicated primary antibodies followed by incubation with the appropriate biotinylated secondary antibodies at 25° C. for 1 h. Pre-immune sera or isotype-matched IgG were routinely included as control. The immunoreaction was developed with DAB as a peroxidase substrate and the sections were counterstained with hematoxylin as for enzyme histochemistry.

### Intestinal Epithelial Cell Isolation

Human colon pieces were recuperated and washed several times with D-PBS containing Fetal Bovine Serum (FBS), antibiotics, antifungal and gentamycin until the solution is clear. Epithelial unit containing stem cells (crypts) were isolated from human samples (as prepared above) or from the intestine of 10-12 weeks old mice by dissection and dissociated with collagenase type I (collagenase XI, DTT and dispase for human). Briefly, the longitudinal muscle layer from the colons were excised, washed with ice-cold Mg²⁺ and Ca²⁺ free Salt Solution (PBS) and then digested with 75 U/mL collagenase type I or collagenase XI. The digestion was stopped with Dulbecco's Modified Eagles Medium (DMEM/F12) containing 10% v/v foetal bovine serum (FBS). L-glutamine, Hepes. N-2 supplement, B-27 supplement and antibiotics. The digestion mixture was filtered with 70 μm filter and the effluent was centrifuged twice at 50×g for 5 min at 4° C. Isolated crypts were cultured on collagen coated-plate or permeable support in DMEM/F12 advanced Medium containing B27, N2 supplement, rmEGF, rmR-spondin1, Noggin, Wnt-3a in presence of Y-27 as anoikis inhibitor. After 48 h, the anoikis inhibitor was removed to form the differentiated IEC monolayer^{52,53,59}. Cells were grown on plates for qPCR experiments and on permeable supports depending of the experiment. The above media without Y-27 was changed after 48 h, and the epithelial cells from the crypts were allowed to differentiate and grow to confluence for 4 days to form a monolayer of IEC.

### Ectonucleotidase Activity

For the determination of luminal ATPase activity, the mice were fasted overnight and anesthetized by isoflurane (Dainippon Sumitomo Pharma). The colon was collected and ligated with nylon threads to make a closed intestinal loop. A total of 200 μl PBS solution with 1.5 mM ATP or without (control) was applied luminally with a 29-G needle. The fluid was recovered 20 min later using a 29-G needle. After centrifugation, the supernatants were collected, and the levels of phosphate were determined with the Malachite green assay (Bigonnesse F, 2004).

ATPase and ADPase activities on differentiated intact IEC at day 4 were performed as previously described (Bigonnesse F, 2004). Protein extraction from cell lysates and ectonucleotidase activity were performed as before (Munkonda Minn., 2007).

### Intestinal Epithelial Cell Stimulation

Murine and human differentiated IEC were grown on plates and stimulated with UDP (100 μM) for 5 hours then the expression of KC (for mouse) and CXCL8 (for human) were evaluated by qPCR.

In other experiments, murine IEC were grown on permeable supports, stimulated on the apical chamber with DSS 3% for 6 hours then the P2Y₆ antagonist MRS 2578 (10 μM) was added for the following 18 hours. The supernatant from basolateral chamber was collected and KC secretion was evaluated by Elisa.

### PCR and Real Time Quantitative PCR (RT-qPCR)

Genomic DNA was extracted from the tail of WT and Entpd8^{-/-} mice using Direct PCR Lysis Reagent to perform PCR using specific primers for NTPDase8 gene and neomycin as mentioned above. Total RNA was isolated from mouse colon and unstimulated IEC monolayer with Trizol then RNA was reverse transcribed to generate cDNA. PCR was performed on cDNA from mouse colon using specific primers to verify the absence of NTPDase8 gene in colons. Primers specific for differentiation marker Villin, ALPi, P2Y receptors, ectonucleotidases or cytokines along with SYBR Green Supermix were used for RT-qPCR using life technologies light cycler 7900. Standard curves were used to determine mRNA transcript copy number in individual reactions. Primer sequences for cytokine analysed were purchased from Qiagen.

### Elisa

Supernatants from IEC stimulated for 24 h were centrifuged (1000×g, 10 min, 4° C.) to discard the detached cells. The supernatants were collected and frozen at −80° C. until determination of cytokine concentrations by ELISA kits (R&D Systems), following the manufacturers' instructions.

### Bone Marrow Transplantation

Bone marrow was isolated aseptically from femurs and tibias of 12-week old donor mice and hematopoietic cells were harvested using standard procedures (Battaile, 1999). To prepare recipient mice for transplantation, recipient male C57BL/6 WT or P2ry6-mice were enterally administered with antibiotics (neomycin sulfate at 1.1 g/L and polymixin B sulfate at 167 mg/L) in drinking water for 1 week before whole-body gamma irradiation (12 Gy) in one dose. A total of 10⁷ unfractionated donor bone marrow cells were injected in the tail vein of the recipient mouse which all received antibiotic in drinking water for 3 weeks after transplantation to reduce infection.

To verify the chimerism, GFP cells were isolated from bone marrow of GFP mice then injected in WT and P2ry6^{-/-} mice in the same conditions as above. 8 weeks after bone marrow transplantation, the degree of chimerism was measured by flow cytometry by comparing the percentage of GFP cells to CD45 cells in peripheral blood.

### Intrarectal Injection of P2Y₆ Antagonist or Apyrase

For intrarectal injection mice were anesthetized and stool that may be present in distal colon was removed with pressure by fingertips to the posterior end of the animal. A 5F infant feeding tube catheter was inserted 4 cm (measured from the midway point between the 2 catheter side ports) into the colon. At this point, 100 μL of P2Y₆ antagonist (MRS 2578 CAS number 711019-86-2, 10 μM), apyrase (4 Units/mice) or their vehicles DMSO or PBS respectively was administered slowly. Following the injection of the solution, animals were positioned head-down for 90 sec to avoid loss of the injected solution. Mice were euthanized 8 hr after the last intrarectal injection, tissue and plasma were isolated for experimental outcomes.

### In Vivo Intestinal Permeability Assay

Intestinal permeability was assessed with fluorescein-labeled dextran (FITC-dextran) as described previously with some modification (Ashleigh Hansen, 2013). All mice were administrated by oral gavage with FITC-dextran (60 mg/100 mg body weight of mouse) 4 h after the intrarectal injection of MRS 2578, apyrase or vehicle (DMSO or PBS). Another 4 hours later, mice were euthanized and whole blood was obtained by cardiac puncture. Plasma was isolated and the movement of FITC-flux from colonic lumen into the plasma was measured by fluorometry at 488 nm. Data are expressed as total quantity of dextran-FITC in 100 μl of plasma.

### In Vitro Intestinal Permeability Assay

Intestinal permeability was assessed with fluorescein-labeled dextran (FITC-dextran) as described previously with some modification (Ashleigh Hansen 2013). FITC-dextran (4 kDa) was added apically to IEC monolayers grown on permeable supports (0.4 μm). 4 hours later, samples were taken from the bottom chambers and fluorescence intensity in the samples was measured. Data were expressed as total quantity of dextran-FITC in supernatant.

### Statistical Analysis

Statistical analysis was performed with Graphpad Prism 6 software. For mouse data, we used ANOVA with Bonferronni test for parametric analyses between three or more groups with Dunn's post hoc test for nonparametric analyses. The data are presented as means±S.E.M.

### Example 2

### NTPDase8 is Localized on Intestinal Epithelial Cells

Knowing that NTPDase8 was expressed in the intestine at the mRNA level (Bigonnesse, 2004), we analysed its cellular localisation in colon. High immuno-reactivity was detected on the apical surface of the mouse intestinal epithelium (FIG. 1A). Likewise, NTPDase8 was also immuno-localized in human colonic epithelial cells from intestinal biopsies (FIG. 1B). We later detected the presence of NTPDase8 by qPCR on human IEC, as presented in Example 8 (FIG. 12).

### Example 3

### Generation and Characterization of Entpd8^{-/-} Mice

We hypothesized that NTPDase8 could regulate the inflammation of the intestine in inflammatory settings. To address this hypothesis, knock out mice were generated on C57Bl/6 background as described in the methods (FIG. 2A). Deletion of Entpd8 gene in intestine was first confirmed by PCR (FIG. 2B, C). There was no physical difference between the WT and NTPDase8 deficient mice with respect to general appearance and body weight gain or growth rate etc. To examine the enzymatic activity of NTPDase8, we evaluated the ATPase and ADPase activities by enzyme histochemistry in mouse intestine sections in presence of phosphatases inhibitors. Although both substrates (ATP and ADP) revealed strong nucleotidase activity throughout the intestinal section with both WT and Entpd8^{-/-} mice, the ecto-ATPase and ecto-ADPase activities were intense on the apical surface of WT intestinal epithelial cells whereas these activities were totally absent in Entpd8^{-/-} sections (FIG. 2D). In agreement, the ex-vivo luminal ATPase activity was significantly reduced in Entpd8^{-/-} intestinal ligated loop (FIG. 2E). The protein level of NTPDase8 was also verified by western blot and it was totally absent in Entpd8^{-/-} homogenate colon (data not shown). Interestingly, there was no significant difference in the total ATPase (17.8±0.5 and 15.8±0.8 nmoles/min·mg Pi for WT and Entpd8^{-/-} respectively) and ADPase (10.4±0.3 and 8±0.5 nmoles/min·mgP for WT and Entpd8a respectively) activities in colons homogenates showing that NTPDase8 is responsible for a minor fraction of this activity in the whole intestine but that it is the major ectonucleotidase at the surface of the intestinal epithelium.

### Example 4

### NTPDase8 Deletion Exacerbates Colitis Induced by DSS

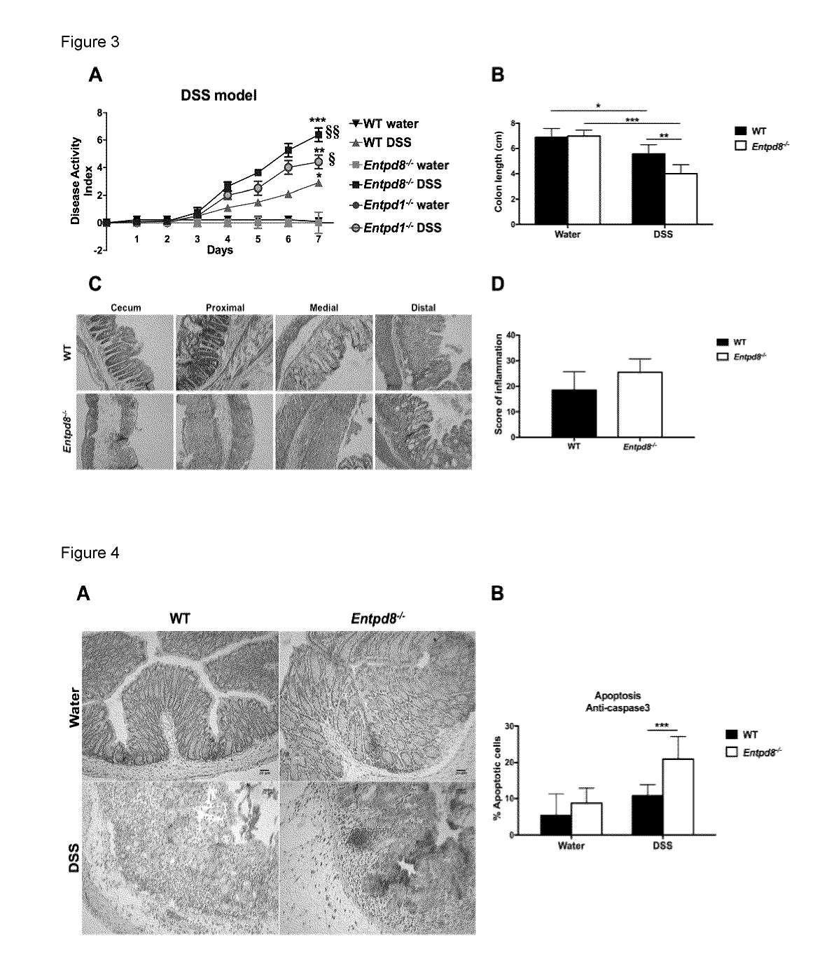

To assess the implication of NTPDase8 in the development of acute colitis, WT and Entpd8^{-/-} mice were exposed to 3% DSS in the drinking water. DSS treatment induced inflammation in these mice as measured by DAI which was dramatically increased in Entpd8^{-/-} mice compared to WT and Entpd1^{-/-} (used as control, Friedman 2009) (FIG. 3A). Moreover, the reduction in colon length, which reflects aggravated intestinal inflammation, was significantly more pronounced in DSS-treated Entpd8^{-/-} mice when compared to WT mice (FIG. 3B). Indeed, DSS treatment induced severe changes in the histology of Entpd8^{-/-} intestines when compared to WT Intestine (FIG. 3C). Nevertheless, this trend didn't reach statistical difference on the histological scoring with the observed criteria (FIG. 3D).

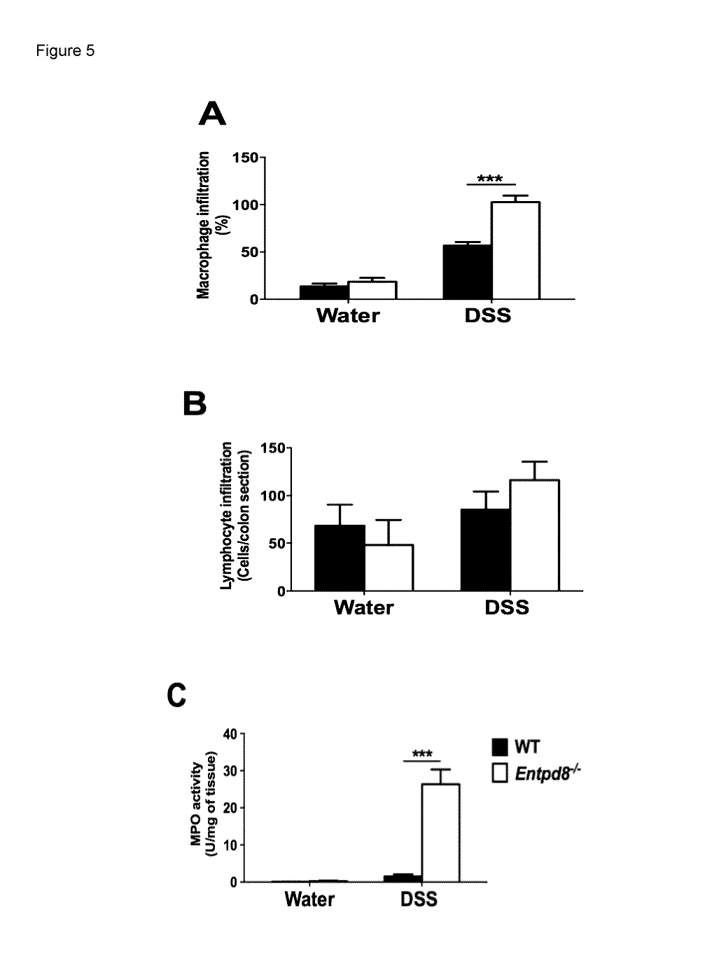

The dramatic inflammation observed in Entpd8^{-/-} mice correlated with increased apoptosis. Indeed, 7 days after DSS treatment, caspase-3 staining was significantly increased in all the regions of Entpd8^{-/-} colons compared to WT mice (FIG. 4). Note that the activity of caspase-3 in colonic sections of control mice showed only a few apoptotic bodies clustered at the top of the epithelium without any difference between Entpd8^{-/-} and WT mice.

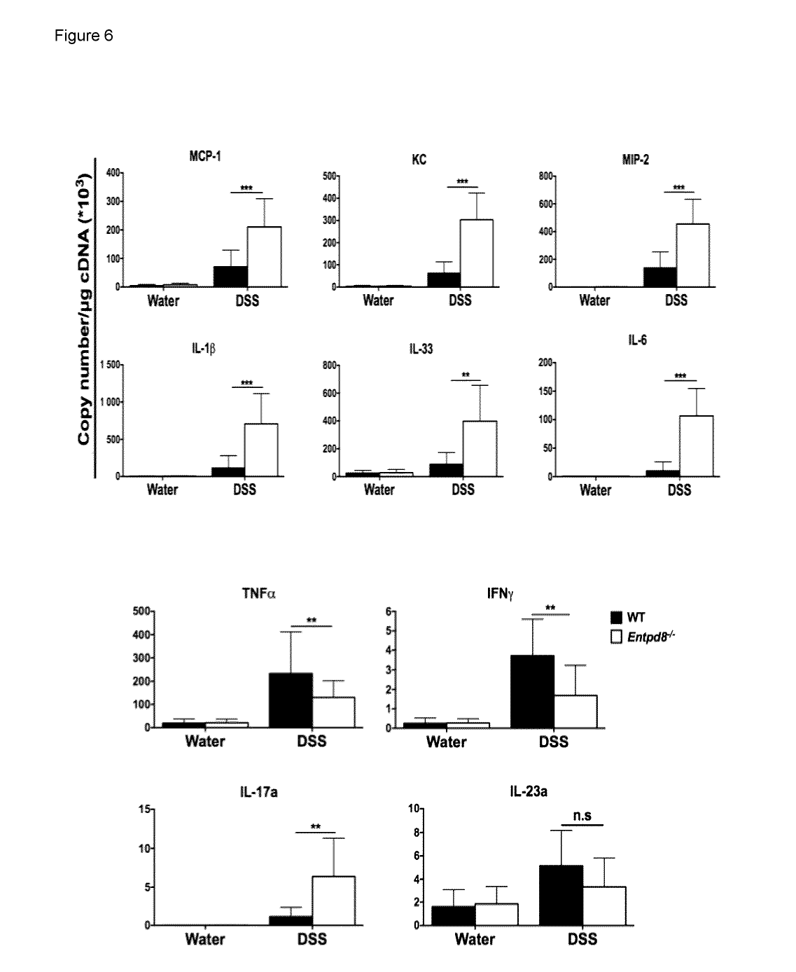

To analyze the inflammatory infiltrate observed in colonic hematoxylin and eosin sections (FIG. 3C) in more details, colon sections were stained with F4/80 and CD3 antibodies, which identify macrophage and lymphocyte infiltration, respectively. A marked increase in the number of F4/80 and a trend for CD3 immunoreactivity was noted in DSS-treated Entpd8^{-/-} mice, which was less prominent in WT intestines (FIGS. 5A and B). Determination of myeloperoxydase enzymatic activity as an index of neutrophil infiltration into intestinal mucosa revealed a 30-fold increase in MPO activity in DSS-treated Entpd8^{-/-} intestines compared to WT intestines (FIG. 5C).

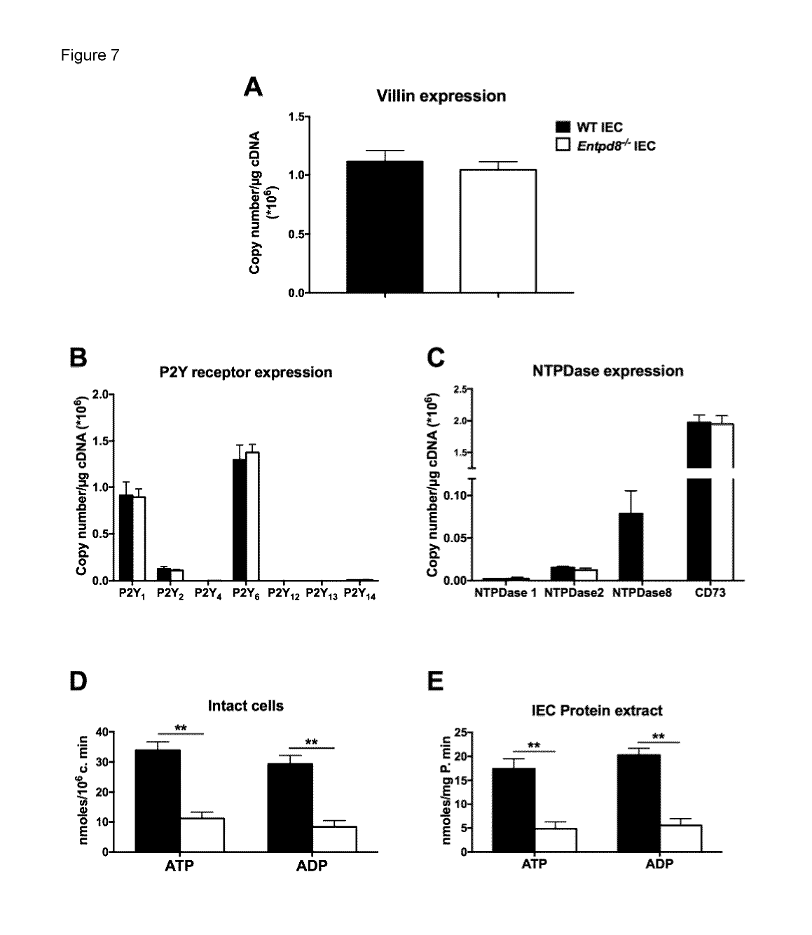

Certain cytokines and proinflammatory mediators contribute to the pathogenicity of IBD and are reportedly increased in DSS-induced colitis. We therefore investigated whether the exacerbation in acute DSS-induced colitis observed in Entpd8^{-/-} mice, was reflected by changes in the expression levels of some of these mediators in colonic tissue homogenates. Upon 7 days of DSS treatment, Entpd8^{-/-} mice showed a significant enhancement in the colonic mRNA levels for chemokines such as macrophage chemoattractant protein-1 (MCP-1), macrophage inflammatory protein 2 (MIP-2) and Keratinocyte chemoattractant (KC) compared to WT mice. DSS treated Entpd8^{-/-} mice also exhibited an increased expression of proinflammatory cytokines such as interleukin-6 (IL-6), IL-1β, IL-33 and IL-17 (FIG. 6). In contrast, the expression of interferon-γ (IFN-γ) and tumor necrosis factor-α (TNF-α) were less expressed in Entpd8^{-/-} when compared to WT mice at this time point.

### Example 5

### Characterization of Murine IEC

The above results show that NTPDase8 plays a crucial role in modulating intestinal inflammation. NTPDase8 is located on the apical surface of IEC. Observation under the microscope did not reveal any difference between WT and Entpd8^{-/-} IEC growth and differentiation showing that these cells can be used for experimentation. In addition, both types of IEC exhibited the same level of villin expression after differentiation, as measured by qRT-PCR (FIG. 7A).

The difference in inflammation observed in Entpd8^{-/-} mice could be due to P2Y receptors over-activation by nucleotides which normally should be hydrolysed by NTPDase8. We therefore measured the expression of P2Y receptors by qRT-PCR and we noted similar and strong amount of P2Y₁, PY₂ and P2Y₆ in both IEC (FIG. 78). We analyzed also the profile of ectonucleotidases by qPCR in IEC and we found that NTPDase8 is the major NTPDase expressed by murine IEC. We noted also high expression of CD73 (FIG. 7C) whose role is to convert AMP to adenosine which has anti-inflammatory properties.

We next evaluated enzymatic activity by malachite green in IEC which showed that intact WT IEC possessed higher ATPase and ADPase activities (34±3 and 29±2 nmoles/10⁶ cells·min, respectively) compared to Entpd8^{-/-} IEC (11.1±1.7 and 8.4±2.2 nmoles/10⁶ cells·min, respectively) (FIG. 7D). Similar results were obtained with protein-extracted from IEC (FIG. 7E).

Together, these data demonstrate the phenotypic similarities between primary IEC from WT and Entpd8^{-/-} mice but a dramatic reduction in ecto-ATPase and ecto-ADPase activities in Entpd8^{-/-} IEC.

### Example 6

The Deletion of P2Y₆ Receptor on Epithelium Protects Entpd8^{-/-} Mice from DSS-Induced Colitis

We hypothesized that the exacerbated inflammation in Entpd8^{-/-} mice was caused by the overactivation of some nucleotide receptors due to the absence of enzyme that regulate their agonist levels. As IEC express P2Y₁, P2Y₂ and P2Y₆ receptors, we suggested to evaluate the role of these receptors on inflammation induced in WT and Entpd8^{-/-} mice. P2Y₁ receptor, expressed on enterochromaffin cells, is implicated in reflex control of intestinal secretion via release of 5-hydroxytryptamine (5-HT) (Cooke H J, 2003). In contrast, P2Y₂ and P2Y₆ receptors have been shown in other cells to exert proinflammatory functions such as to Induce the release of chemokines from leukocytes (Kukulski et al, review 2011). These receptors were also associated with TNF/IFN release in the human Intestinal cell line Caco-2 (Grbic DM, 2008). We therefore tested the effect of the deletion of these genes in the DSS model.

As P2ry6^{-/-} mice did not show resolution of Inflammation in the DSS model (data not shown) and given that P2Y₆ receptor is also expressed in myeloid cells (Somers G R, 1998), we generated bone marrow chimeric mice to discriminate its role in non-hematopoietic cells (e.g. IEC). Chimeras generated were noted as follows (donor->recipient) WT->P2ry6^{-/-} and P2ry6^{-/-}->WT. Control chimeras were also generated WT->WT and P2ry6^{-/-}->P2ry6^{-/-}. Then, mice were subjected to DSS-induced inflammation. First, chimerism was assessed by the presence of GFP cells in the circulating peripheral blood 8 weeks following the transplantation of bone marrow. No significant differences in the number of circulating GFP⁺ cells in the peripheral blood was observed after the transplantation in WT and P2ry6^{-/-} mice (FIG. 8A). Accordingly, WT and P2ry6^{-/-} mice were reconstituted similarly after irradiation. We could then evaluate the inflammation Induced by DSS in these chimeras. In contrast to other chimeras, mice deficient for P2Y₆ receptor in IEC (WT->P2ry6^{-/-}) were totally protected from colitis after DSS treatment while the 3 other groups of chimeras (WT->WT, P2ry6^{-/-}->WT and P2ry6^{-/-}->P2ry6^{-/-}) had all very high levels of the 3 indicators of inflammation measured, namely high DAI, high myeloperoxydase activity and reduced colon length. In contrast, these 3 parameters in WT->P2ry6^{-/-} DSS-treated mice were similar to the control mice that received water without DSS (FIG. 8B-D). These results are in agreement with histopathological analysis (FIG. 8E) where severe histological changes in DSS-induced colitis were observed in intestine of WT->WT, P2ry6^{-/-}->WT and P2ry6′->P2ry6^{-/-} with crypt damage and infiltration of inflammatory cells whereas intestines of WT->P2ry6^{-/-} chimera showed total absence of inflamed area and were similar to mice that received water. The quantification of the profile of cytokines and chemokines expressed in DSS-treated colons of the different chimeras showed that WT->P2ry6^{-/-} chimera exhibited lower expression levels of proinflammatory cytokines compared to the 3 other chimeras (FIG. 8F). Collectively, these data demonstrate a major role of P2Y₆ receptor expressed on IEC in intestinal inflammation and that its deletion totally prevents intestinal inflammation in mice in the DSS model.

Next, we used P2ry2^{-/-} mice to identify the role of P2Y₂ receptors in intestinal inflammation in vivo. Acute DSS treatment of P2ry2^{-/-} mice showed lower DAI level compared to WT mice (FIG. 9) suggesting that P2Y₂ receptor could also affect intestinal inflammation similarly as P2Y₆ receptor but to a lesser extent.

### Example 7

### Intrarectal Injection of Apyrase or P2Y₆ Antagonist Ameliorates DSS-Induced Colitis

The above results obtained from bone marrow transplantation showing that the deletion of P2Y₆ receptor specifically on IEC completely protected intestinal inflammation suggest that the increased inflammation observed in the same model in Entpd8^{-/-} mice would be explained by the fact that NTPDase8, normally present on IEC, reduces the agonist levels of P2Y₆, and therefore controls the level of its activation. We therefore tested whether the pharmacological blockade of P2Y₆ in Entpd8^{-/-} mice treated with DSS could lower the inflammation in these mice. These data also suggest that the pharmacological inhibition of P2Y₆ could be used therapeutically to reduce Intestinal inflammation. In agreement, the daily injection of P2_{Y} antagonist (MRS 2578, 10 μM) intrarectally to both WT and Entpd8^{-/-} mice from day 2 to day 7 of DSS treatment reduced inflammation and the DAI when compared with mice that received DSS alone (FIG. 10A, C). In addition, blocking the P2Y₆ receptor with MRS 2578 significantly ameliorated the permeability as assessed by FITC-dextran flux from intestine to plasma (FIG. 10B, D).

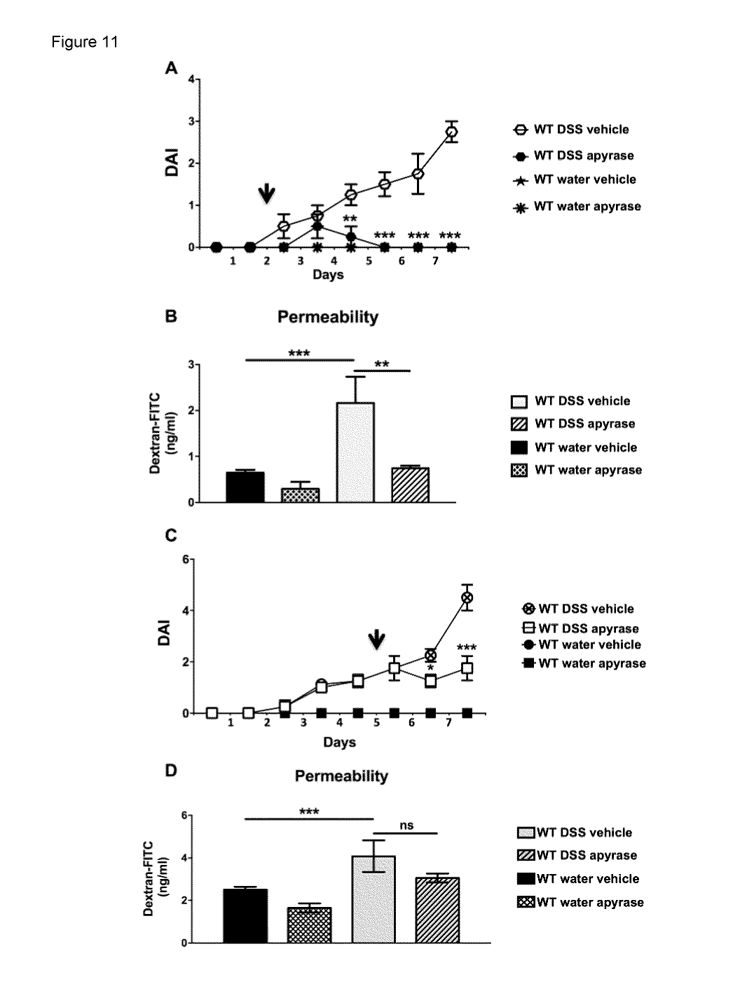

Considering that NTPDase8 is important to hydrolyse nucleotides released in the Intestinal lumen upon DSS treatment, we next proposed to recover the Intestinal inflammation of WT mice by injecting NTPDase8 nucleotidase-like activity directly in the Intestinal lumen to add further nucleotidase activity to the NTPDase8 already present in WT IEC. This would therefore result in blockade of P2Y₂ and P2Y₆ receptors. As shown in FIGS. 11A and B, the daily administration of apyrase, an enzyme with similar activity as NTPDase8, from day 2 to day 7 of DSS treatment fully protected WT mice from colitis as determined by DAI and permeability measurements. Control mice were injected with vehicle (PBS) only. Moreover, the injection of apyrase in late phase of DSS treatment (from day 5 to day 7) also reduced inflammation as seen by a decreased in the DAI, again when compared to WT mice that received the vehicle (PBS) only (FIGS. 11C, D).

### Example 8

### Characterization of Human IEC

According to our results above with murine IEC, NTPDase8 and P2Y₆ receptor are the major ectonucleotidase and receptor expressed on IEC and they play a crucial role in modulating intestinal inflammation in mice. Next, we analyzed the profile of ectonucleotidases and P2Y receptors in human IEC. The normal differentiation of IEC from the crypts was confirmed by the increase of villin and ALPi markers (FIG. 12A). Concerning the expression of ectonucleotidases and P2Y receptors, similar profile as for murine IEC were obtained. NTPDase8 was the major ectonucleotidase (FIG. 12B) and P2Y₆ was the major P2Y receptor (FIG. 12C).

### Example 9

### P2Y₆ Activation Increased Chemokine Expression

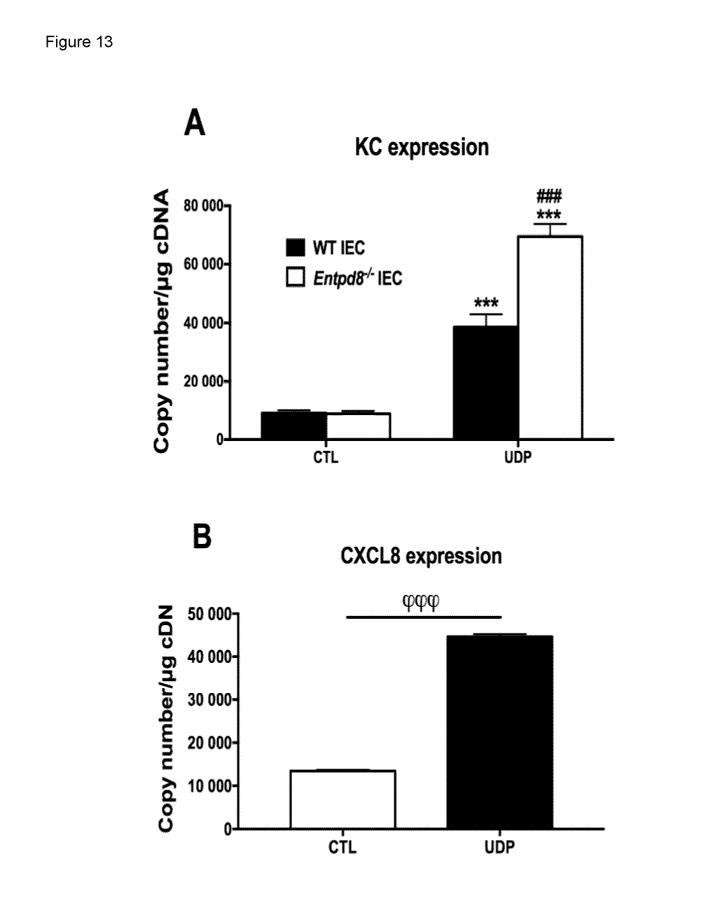

To verify the role of P2Y₆ receptor on IEC function, murine and human IEC were stimulated with UDP as the specific ligand for P2Y₆ receptor. Our results show the implication of UDP in cytokine expression as we noted significant increase of KC and CXCL8 expression upon UDP stimulated murine and human IEC, respectively. We noted also a higher amount of KC in IEC deficient for NTPDase8 than WT (FIG. 13) which correlate with the dramatic increase of inflammation in Entpd8^{-/-} mice in DSS model, obviously due to an overactivation of P2Y₆ (FIG. 12). These data also confirm that the P2Y₆ that we observed in IEC of both mouse and human IEC is functional and that it can regulate the secretion of proinflammatory cytokines.

### Example 10

### Blocking P2Y₆ Receptor Blocks Proinflammatory In Vitro Effects

As shown above, DSS treatment in vivo induced intestinal inflammation in mice. To mimic these inflammatory conditions in vitro, differentiated epithelial cells from WT and Entpd8^{-/-} mice were treated with DSS 3% for 24 hours then intestinal permeability together with KC secretion were evaluated. We showed that DSS treatment increased permeability in both IEC genotypes when compared to their respective control. More importantly, Entpd8^{-/-} IEC monolayer was more permeable than WT IEC suggesting the implication of nucleotide release in these phenomena. In agreement with this result, KC secretion was more importantly released by Entpd8^{-/-} IEC than by WT IEC after DSS treatment (FIG. 14). Giving the fact that P2Y₆ is the major receptor expressed on IEC, we next repeated those tests in presence of a P2Y₆ receptor antagonist which reduced as expected both of these effects even if the antagonist was added 6 hours after DSS (FIG. 14).

### Example 11

### Discussion

This research shows that NTPDase8 is localized on the apical side of mice colonic epithelium and that it is responsible for its ATPase and ADPase activities. Our results suggest that NTPDase8 plays an important role in protecting the intestinal epithelium as the administration of the NTPDase8-like apyrase protected mice from inflammation in the DSS-induced experimental colitis model, via limiting P2Y₆ receptor activation as demonstrated by bone marrow transplantation (BMT) studies and intrarectal Injection of antagonist and apyrase. Therefore, we herein propose that extracellular nucleotide hydrolysis is a key process to prevent Intestinal inflammation. In agreement, we observed that in the absence of NTPDase8 in Entpd8^{-/-} mice, these mice were dramatically more susceptible to DSS-induced colitis as seen by Increased DAI, shortened colons, increased infiltration of macrophages and neutrophils as well as an increased expression of several proinflammatory cytokines. The deletion of NTPDase8 in colon also contributed to increase apoptosis in the intestine of DSS treated mice which may be the consequence of acute damage (Araki Y, 2010).

The increased macrophage infiltration observed in DSS treated Entpd8^{-/-} mice correlate with a characteristic feature of IBD which is presumed to have a key role in IBD pathogenesis and DSS induced colitis (Zigmond, E. 2013; Rose L, 2013). It was suggested that resident intestinal macrophages, when activated, are responsible of chemokine release into body fluid which then favor the recruitment of effector immune cells such as monocytes and neutrophils (Mogensen T H, 2009). In agreement, Entpd8^{-/-} intestines of DSS treated mice expressed higher mRNA levels of KC, MIP-2 and MCP-1, which are known to trigger the infiltration of neutrophils and macrophages, respectively (Mihaescu A 2010, Singh UP 2016).

NTPDase8, expressed on the epithelium, may play its protective role by decreasing the agonist levels of P2 receptors, thereby, limiting the activation of these nucleotide receptors at the surface of IEC. We measured similar expression levels of P2Y₁, P2Y₂ and P2Y₆ In both WT and Entpd8^{-/-} IEC. Those receptors may therefore be overactivated in the absence of NTPDase8 in Entpd8^{-/-} mice. We focused our attention on P2Y₂ and P2Y₆ receptors since they have been previously associated with proinflammatory functions in other cells such as leukocytes (Kukulski et al, 2011). Of specific interest, P2Y₆ activation was also reported to induce the release of the proinflammatory cytokine CXCL-8 in the intestinal cell line Caco-2 (Grbic D M, 2008).

As P2Y₆ receptor is expressed on both leukocytes and IEC, we did BMT studies to discriminate their effects on intestinal inflammation and more importantly to address specifically the function of P2Y₆ expressed on IEC. Of great therapeutic interest, P2ry6^{-/-} mice that received bone marrow from WT mice were fully protected from inflammation in the DSS colitis model. Indeed, they had a DAI, a colon length, MPO activity as well as a histopathology comparable to mice that did not receive DSS. In addition, the expression levels of proinflammatory cytokines at day 7 were also dramatically reduced in mice deficient in P2ry6^{-/-} in their IEC when compared to the other control mice that received DSS. Overall these data confirm that the intestinal epithelium plays a key role in inflammation of the bowel and that P2Y₆ expressed in these cells in deeply involved in this process.

As mentioned above we estimated that P2Y₂ could also be involved in the inflammation observed in DSS treated mice. Therefore, we tested mice deficient in P2Y₂ In this colitis model. As shown in FIG. 9, P2ry2^{-/-} mice were partially protected from colitis suggesting that P2Y₂ could also play a role in IBD but, presumably, to a lower extent than P2Y₆.

In light of this data, our study demonstrates that NTPDase8 localised on the apical surface of the intestinal epithelium reduces intestinal inflammation by preventing the activation of P2Y₆ and P2Y₂ receptors in these cells. These data suggest that either the implementation of NTPDase8 activity in the lumen of the bowel and/or the blockade of P2Y₆ and P2Y₂ receptors on the intestinal epithelium could be used to treat IBD in human.

This hypothesis was tested in the same model in mice. We injected intrarectally an antagonist of P2Y₆ as well as apyrase as a NTPDase8-like enzyme available commercially. As expected we observed that the blockade of P2Y₆ receptor activation in Entpd8a as well as in WT mice attenuated inflammation in the DSS experimental colitis model. In addition, the hydrolysis of nucleotides in the lumen of the intestine by apyrase protected totally WT mice from DSS induced colitis (FIG. 11).

Applicants believe that these observations in mice are also valid in human because they have demonstrated in FIG. 1 the presence of NTPDase8 at the surface of human intestinal epithelial cells and that P2Y₂ and P2_{Y} have also been reported to be expressed in Caco-2 which is a human Intestinal cell line (Emilie Degagné, 2009; Grbic, 2008). Taken together, the data presented here demonstrate the primary role of nucleotides signalling at the surface of the intestinal epithelium in the inflammation of this tissue. Most importantly that blocking P2Y₂ and P2Y₆ receptors pharmacologically, genetically or by the supplementation of NTPDase8 activity in the lumen of the intestine protects from inflammation suggesting that this pathway can be used to treat IBD in humans.

Since the main interest was to find a new therapeutic target for human intestinal inflammation, Applicants wanted to further confirm the findings with mice in human. As previously mentioned, the expression of some P2Y receptors expression have been evaluated in cell lines but to date, many differences in epithelial with cell lines have been observed (Grbic D M, 2008; Bahrami F, 2014) and there was no study carried out with human primary cells which are the real human cells. Therefore, we characterized the members of purinergic signaling in primary IEC isolated form human colon samples. Here, we showed that human IEC expressed predominantly the ectonucleotidase NTPDase8 and the P2Y₆ nucleotide receptor. Lower expressions were also noted for P2Y₁ and P2Y₂ receptors than what observed in mice. We also verified whether blocking P2Y₆ could have similar effects in human. Our first interest was to evaluate in vitro the activity of P2Y₆ receptor using primary murine and human IEC. Stimulation of P2Y₆ with its natural specific ligand UDP increased KC and CXCL8 expression in murine and human IEC, respectively, demonstrating that P2Y₆ is active in IEC and participates in their Immune function as chemokine expression. These results are in agreement with previous studies in which Grbic et al. has shown with a human epithelial derived cell line, that these immortal cells secreted CXCL8 upon UDP stimulation. Our in vitro assays when blocking P2Y₆ receptor in vitro in inflammatory condition with murine IEC resulted in a decreased permeability and KC release showing 2 different proinflammatory aspects that P2Y₆ can control which is in agreement with the fact that P2Y₆ blockade could be a therapeutic treatment for IBD. Therefore, these data also support our initial hypothesis that P2Y₆ receptor could be a therapeutic target for IBD. In perspective, it will be interesting to investigate the mechanism regulated by P2Y₆ receptor responsible of protection against intestinal inflammation. Furthermore, determine the role of P2Y₆ receptor on other epithelial cell functions such as integrity controlled by tight junctions which may be also implicated in symptoms related to intestinal inflammation.

### CONCLUSION

We have demonstrated that NTPDase8 is localized on the apical side of colonic epithelium and that its deletion exacerbates Intestinal inflammation by promoting the activation of P2Y₆ receptor expressed on IEC. We showed also that P2Y₆ deficiency in murine IEC in vivo exerts a protective role on intestinal inflammation development and that the full deletion of P2Y₂ receptor also protects mice partially from DSS-induced colitis. In addition, it has been demonstrated in vitro that activation of P2_{Y} results in chemokine expression in murine and human IEC and that blocking it reduced epithelial permeability and chemokine secretion, which are two proinflammatory effects.

The present study suggests that the regulation of nucleotide signalling by NTPDase8 in IEC could control the inflammation by controlling the agonist level of P2Y₂ and P2Y₆ receptors.

While the invention has been described in connection with specific embodiments thereof, it will be understood that it is capable of further modifications and this application is intended to cover any variations, uses, or adaptations of the invention following, in general, the principles of the invention and Including such departures from the present disclosure as come within known or customary practice within the art to which the invention pertains and as may be applied to the essential features herein before set forth, and as follows in the scope of the appended claims.

All patents, patent applications and publications mentioned in this specification are herein incorporated by reference to the same extent as if each independent patent, patent application or publication was specifically and individually indicated to be incorporated by reference.

### REFERENCES

Xu X R, Liu C Q, Feng B S, Liu Z J. Dysregulation of mucosal immune response in pathogenesis of inflammatory bowel disease. World J Gastroenterol. 2014 Mar. 28; 20(12):3255-64.
Lakhan S E, Kirchgessner A. Neuroinflammatlon in Inflammatory bowel disease. J Neuroinflammation. 2010 Jul. 8; 7:37.
Kono H, Rock K L. How dying cells alert the immune system to danger. Nat Rev Immunol. 2008 April; 8(4):279-89.
Lister M F, Sharkey J, Sawatzky D A, Hodgkiss J P, Davidson D J, Rossi A G, Finlayson K. The role of the purinergic P2X7 receptor in inflammation. J Inflamm (Lond). 2007 Mar. 16; 4:5.
Riteau N, Gasse P, Fauconnier L, Gombault A, Couegnat M, Fick L, Kanellopoulos J, Quesniaux V F, Marchand-Adam S, Crestani B, Ryffel B, Couillin I. Extracellular ATP is a danger signal activating P2X7 receptor in lung inflammation and fibrosis. Am J Respir Crit Care Med. 2010 Sep. 15; 182(6):774-83.
Antonioli L, Fornai M, Colucci R, Ghisu N, Tuccori M, Del Tacca M, Blandizzi C; Pharmacological modulation of adenosine system: novel options for treatment of inflammatory bowel diseases; Inflamm Bowel Dis. 2008; 14 (4):566-74. Review.
Antonioli L, Colucci R, Pellegrini C, Giustarini G. Tuccori M. Blandizzi C, Fornai M. The role of purinergic pathways in the pathophysiology of gut diseases: pharmacological modulation and potential therapeutic applications. Pharmacol Ther. 2013 August; 139(2):157-88.
Grbic D M, Degagné E, Langlois C, Dupuis A A, Gendron F P; Intestinal inflammation increases the expression of the P2Y6 receptor on epithelial cells and the release of CXC chemokine ligand 8 by UDP; J Immunol. 2008 Feb. 15; 180 (4):2659-68.
Burnstock G: Purine and pyrimidine receptors. Cell Mol Life Sci. 2007 June: 64 (12):1471-83. Review.
Bigonnesse F, Lévesque S A, Kukulski F, Lecka J, Robson S C, Femandes M J, Sévigny Jean; Cloning and characterization of mouse nucleoside triphosphate diphosphohydrolase-8; Biochemistry. 2004 May 11; 43(18):5511-9.
Deaglio S1, Dwyer K M, Gao W, Friedman D, Usheva A, Erat A, Chen J F, Enjyoji K, Linden J, Oukka M, Kuchroo V K, Strom T B, Robson S C. Adenosine generation catalyzed by CD39 and CD73 expressed on regulatory T cells mediates immune suppression. J Exp Med. 2007 Jun. 11; 204(6):1257-65. Epub 2007 May 14.
Friedman D J, Künzli B M, A-Rahim Y I, Sevigny J, Berberat P O, Enjyoji K, Csizmadia E, Friess H, Robson S C; From the Cover: CD39 deletion exacerbates experimental murine colitis and human polymorphisms increase susceptibility to inflammatory bowel disease; Proc Natl Acad Sci USA. 2009 Sep. 29; 106(39):16788-93.
Buergler J M, Maliszewski C R, Broekman M J, Kaluza G L, Schulz D G, Marcus A J, Raizner A E, Kleiman N S, Ali N M. Effects of SolCD39, a novel inhibitor of Platelet Aggregation, on Platelet Deposition and Aggregation after PTCA in a Porcine Model. J Thromb Thrombolysis. 2005 April; 19(2):115-22.
Gayle R B 3rd, Maliszewski C R, Gimpel S D, Schoenborn M A, Caspary R G, Richards C. Brasel K, Price V, Drosopoulos J H. Islam N. Alyonycheva T N. Broekman M J, Marcus A J. Inhibition of platelet function by recombinant soluble ecto-ADPase/CD39. J Clin Invest. 1998 May 1; 101(9); 1851-9.
Berberat P O, A-Rahim Y I, Yamashita K, Warny M M, Csizmadia E, Robson S C, Bach F H; Heme oxygenase-1-generated biliverdin ameliorates experimental murine colitis; Inflamm Bowel Dis. 2005 April; 1l (4):350-9.
Laroui H, Sarah A. Ingersol, Hong Chun Liu, Mark T. Baker, Saravanan Ayyadural, Moiz A. Charania, Famina Laroui2, Yutao Yen, Shanthi V. Sitearaman, Didier Merlin; Dextran Sodium Sulfate (DSS) Induces Colitis in Mice by Forming Nano-Lipocomplexes with Medium-ChainLength Fatty Acids in the Colon; PLoS ONE; 2012: Volume 7, Issue 3,e32084
Martin-Satué M, Lavoie E G, Pelletier J, Fausther M, Cslzmadia E, Guckelberger O, Robson S C, Sévigny J Localization of plasma membrane bound NTPDases in the murine reproductive tract; Histochem Cell Biol. 2009 May; 131(5):615-28.
Moon C, VanDussen K L, Miyoshi H, Stappenbeck T S; Development of a primary mouse intestinal epithelial cell monolayer culture system to evaluate factors that modulate IgA transcytosis; Mucosal Immunol. 2014 July; 7(4):818-28.
Graves C L. Harden S W, LaPato M, Nelson M. Amador B, Sorenson H. Frazier C J, Wallet S M; A method for high purity intestinal epithelial cell culture from adult human and murine tissues for the investigation of innate immune function; J Immunol Methods. 2014 Dec. 1; 414:20-31.
Munkonda M N. Kauffenstein G, Kukulski F, Lévesque S A, Legendre C, Pelletier J. Lavoie E G, Lecka J, Sévigny J; Inhibition of human and mouse plasma membrane bound NTPDases by P2 receptor antagonists; Biochem Pharmacol. 2007 Nov. 15; 74(10):1524-34.
Battaile K P, Bateman R L, Mortimer D, Mulcahy J, Rathbun R K, Bagby G, Fleming W H, Grompe M: In vivo selection of wild-type hematopoietic stem cells in a murine model of Fanconi anemia; Blood. 1999 Sep. 15; 94(6): 2151-8.
Hansen A, Alston L, Tulk S E, Schenck L P, Grassie M E, Alhassan B F, Veermalla A T, A I-Bashir S, Gendron F P, Altier C, MacDonald J A, Beck P L, Hirota S A. The P2Y₆ receptor mediates Clostridlum difficile toxin-induced CXCL8/IL-8 production and intestinal epithelial barrier dysfunction. PLoS One. 2013 Nov. 22; 8(11):e81491.
Cooke H J, Wunderlich J, Christofi F L; The force be with you”: ATP in gut mechanosensory transductionb News Physiol Sci. 2003 April; 18:43-9.
Kukulski F, Lévesque S A. Sévigny J. Impact of ectoenzymes on P2 and P1 receptor signaling. Adv Pharmacol. 2011; 61:263-99.
Beaudoin A R, Sévigny J. Picher M. (1996) ATP-diphosphohydrolases, apyrases, and nucleotide phosphohydrolases: biochemical properties and functions. In ATPases. (Lee A.G., ed.), Biomembranes Vol. 5, pp. 369-401. Greenwich, C T. JAI Press Inc.
Somers G R, Hammet F M, Trute L, Southey M C, Venter D J. Expression of the P2Y₆ purinergic receptor in human T cells infiltrating inflammatory bowel disease; Lab Invest. 1998 November; 78(11):1375-83.
Araki O, Kenichi Mukaisyo, Hiroyuki Sugihara, Yoshihide Fujiy Ama and Akanori T Hattori. Increased apoptosis and decreased proliferation of colonic epithelium in dextran sulfate sodium-induced colitis in mice. Oncology reports 24: 869-874, 2010
Zigmond E, Jung S; Intestinal macrophages: well educated exceptions from the rule. Trends Immunol. 2013 April; 34(4):162-8. Review.
Rose L. Szabady, and Beth A. McCormick; Control of Neutrophil Inflammation at Mucosal Surfaces by Secreted Epithelial Products; Front Immunol. 2013; 4: 220.
Mogensen T H; Pathogen recognition and inflammatory signaling in innate immune defenses; Clin Microbiol Rev. 2009 April; 22(2):240-73
Mihaescu A, Santen S, Jeppsson B, Thorlacius H. p38 Mitogen-activated protein kinase signalling regulates vascular inflammation and epithelial barrier dysfunction in an experimental model of radiation-induced colitis. Br J Surg. 2010 February; 97(2):226-34.
Singh U P, Sing N P, Murphy E A, Price R L, Fayad R, Nagarkatti M, Nagarkatti P S. Nagarkatti P S; Chemokine and cytokine levels in inflammatory bowel disease patients Cytokine 77 (2016) 44-49
Gil V, Martlnez-Cutillas M, Mañé N. Martin M T, Jiménez M. Gallego D. P2Y₁ knockout mice lack purinergic neuromuscular transmission in the antrum and cecum. Neurogastroenterol Motil. 2013 March; 25(3):e170-82.
Hwang S J, Blair P J, Dumin L, Mutafova-Yambolieva V. Sanders K M, Ward S M; P2Y₁ purinoreceptors are fundamental to inhibitory motor control of murine colonic excitability and transit; J Physiol. 2012 Apr. 15:590(Pt 8):1957-72.
Gallego D, Gil V, Martinez-Cutillas M, Mañé N, Martin M T, Jiménez M; Purinergic neuromuscular transmission is absent in the colon of P2Y1 knocked out mice; J Physiol. 2012 Apr. 15: 590(Pt 8):1943-56.
Kukulski F. Bahrami F, Ben Yebdri F, Lecka J, Martin-Satué M, Lévesque S A, Sévigny J. NTPDase1 controls IL-8 production by human neutrophils. J Immunol. 2011 Jul. 15; 187(2):644-53.
Degagné E. Grbic D M. Dupuis A A, Lavoie E G. Langlois C, Jain N, Weisman G A, Sévigny J, Gendron F P. P2Y₂ receptor transcription is increased by N F-kappa B and stimulates cyclooxygenase-2 expression and PGE2 released by intestinal epithelial cells. J Immunol. 2009 Oct. 1:183(7):4521-9.

What is claimed is:

1-23. (canceled)

24. A method for the treatment or prevention of a chronic inflammatory bowel disease in a mammal, the method comprising administering to said mammal an effective amount of a composition comprising a Nucleoside triphosphate diphosphohydrolase (NTPDase) enzyme, wherein the enzyme is selected from the group consisting of: NTPDase3 and NTPDase8.

25. The method of claim 24, wherein said NTPDase enzyme is in a concentration sufficient to decrease a concentration of nucleoside triphosphate or diphosphate in a colon lumen of said mammal.

26. The method of claim 25, wherein said nucleoside triphosphate or diphosphate is selected from the group consisting of UTP, UDP, ADP and ATP.

27. The method of claim 25, wherein said composition further comprises an inhibitor or antagonist of P2Y₆ receptor.

28. The method of claim 25, wherein said composition further comprises an inhibitor or antagonist of P2Y₂ receptor.

29. The method of claim 25, wherein the administering comprises administering orally, enterally or intrarectally.

30-42. (canceled)

43. The method according to claim 24, wherein said inflammatory bowel disease is selected from the group consisting of Crohn's disease, ulcerative colitis and GI tract cancer.

44-49. (canceled)

50. The method according to claim 24, wherein said enzyme is NTPDase8.


SEQUENCE LISTING (4 sequences)

SEQ ID NO: 1 - DNA, 20 bp, Mus musculus
tgtaagggccagaaggattg

SEQ ID NO: 2 - DNA, 20 bp, Mus musculus
gtacagacccgaggcacagt

SEQ ID NO: 3 - DNA, 22 bp, Mus musculus
tcattctcagtattgttttgcc

SEQ ID NO: 4 - DNA, 20 bp, Mus musculus
caggcatctaggatctgctc